SEQUENCE LISTING (98 sequences)

SEQ ID NO: 1 - protein, 9 aa, synthetic construct
LLLEATANL

SEQ ID NO: 2 - protein, 9 aa, synthetic construct
SLTDLYLRI

SEQ ID NO: 3 - protein, 9 aa, synthetic construct
IMTASNWTL

SEQ ID NO: 4 - protein, 9 aa, synthetic construct
AMSPQLHNI

SEQ ID NO: 5 - protein, 9 aa, synthetic construct
GLAAPAPKL

SEQ ID NO: 6 - protein, 9 aa, synthetic construct
GLPPHPAHL

SEQ ID NO: 7 - protein, 9 aa, synthetic construct
GMPWPIPAV

SEQ ID NO: 8 - protein, 9 aa, synthetic construct
SLQETSYAL

SEQ ID NO: 9 - protein, 9 aa, synthetic construct
SLYPIACSL

SEQ ID NO: 10 - protein, 9 aa, synthetic construct
SVLGHDFSV

SEQ ID NO: 11 - protein, 9 aa, synthetic construct
VQDMIQTQV

SEQ ID NO: 12 - protein, 9 aa, synthetic construct
ILDDWLRHL

SEQ ID NO: 13 - protein, 9 aa, synthetic construct
SLPSQHWSL

SEQ ID NO: 14 - protein, 9 aa, synthetic construct
FLLMPLSFL

SEQ ID NO: 15 - protein, 9 aa, synthetic construct
IILGIVFLL

SEQ ID NO: 16 - protein, 9 aa, synthetic construct
SLDHLLLEA

SEQ ID NO: 17 - protein, 9 aa, synthetic construct
FLWKRGRLL

SEQ ID NO: 18 - protein, 9 aa, synthetic construct
ALLDGVLPA

SEQ ID NO: 19 - protein, 9 aa, synthetic construct
LLFKVDFFL

SEQ ID NO: 20 - protein, 9 aa, synthetic construct
RLGAAVFLL

SEQ ID NO: 21 - protein, 9 aa, synthetic construct
RLLAKGQSL

SEQ ID NO: 22 - protein, 9 aa, synthetic construct
SQPPPSLFV

SEQ ID NO: 23 - protein, 9 aa, synthetic construct
ALLRCGHTL

SEQ ID NO: 24 - protein, 9 aa, synthetic construct
GQASVPLFL

SEQ ID NO: 25 - protein, 9 aa, synthetic construct
KLQTLLASI

SEQ ID NO: 26 - protein, 9 aa, synthetic construct
LLASILFYI

SEQ ID NO: 27 - protein, 9 aa, synthetic construct
LLVSTGVTV

SEQ ID NO: 28 - protein, 9 aa, synthetic construct
QMQPHNLGV

SEQ ID NO: 29 - protein, 9 aa, synthetic construct
SLFKLQTLL

SEQ ID NO: 30 - protein, 9 aa, synthetic construct
VMFFKSQHL

SEQ ID NO: 31 - protein, 9 aa, synthetic construct
ALADEWRNL

SEQ ID NO: 32 - protein, 9 aa, synthetic construct
ALMISLGSV

SEQ ID NO: 33 - protein, 9 aa, synthetic construct
ALWQDHTEI

SEQ ID NO: 34 - protein, 9 aa, synthetic construct
AQWPAPRLV

SEQ ID NO: 35 - protein, 9 aa, synthetic construct
CLLSKPVRL

SEQ ID NO: 36 - protein, 9 aa, synthetic construct
FLFSICKQL

SEQ ID NO: 37 - protein, 9 aa, synthetic construct
FLLTPRNFL

SEQ ID NO: 38 - protein, 9 aa, synthetic construct
FMMITAYTV

SEQ ID NO: 39 - protein, 9 aa, synthetic construct
FSIELLFSV

SEQ ID NO: 40 - protein, 9 aa, synthetic construct
GLFFSLMFL

SEQ ID NO: 41 - protein, 9 aa, synthetic construct
GLKPWTQYA

SEQ ID NO: 42 - protein, 9 aa, synthetic construct
KLFGFCFQL

SEQ ID NO: 43 - protein, 9 aa, synthetic construct
KLISELRRI

SEQ ID NO: 44 - protein, 9 aa, synthetic construct
KLSELLMSF

SEQ ID NO: 45 - protein, 9 aa, synthetic construct
LLFSVNREV

SEQ ID NO: 46 - protein, 9 aa, synthetic construct
LLLAGGPGL

SEQ ID NO: 47 - protein, 9 aa, synthetic construct
LLPGGLLLL

SEQ ID NO: 48 - protein, 9 aa, synthetic construct
LLPPAPLVV

SEQ ID NO: 49 - protein, 9 aa, synthetic construct
LLQALMISL

SEQ ID NO: 50 - protein, 9 aa, synthetic construct
LLVISLWSV

SEQ ID NO: 51 - protein, 9 aa, synthetic construct
LLWKLISEL

SEQ ID NO: 52 - protein, 9 aa, synthetic construct
QIIQLVIRV

SEQ ID NO: 53 - protein, 9 aa, synthetic construct
RLAPLFQQL

SEQ ID NO: 54 - protein, 9 aa, synthetic construct
SLKDGVFTT

SEQ ID NO: 55 - protein, 9 aa, synthetic construct
SLSWETPGV

SEQ ID NO: 56 - protein, 9 aa, synthetic construct
SLWPHPTTV

SEQ ID NO: 57 - protein, 9 aa, synthetic construct
SMMGRMPAA

SEQ ID NO: 58 - protein, 9 aa, synthetic construct
SVHPTAPAV

SEQ ID NO: 59 - protein, 9 aa, synthetic construct
SVPKHVWEA

SEQ ID NO: 60 - protein, 9 aa, synthetic construct
VLFKLSELL

SEQ ID NO: 61 - protein, 9 aa, synthetic construct
VLFSILVST

SEQ ID NO: 62 - protein, 9 aa, synthetic construct
ALLTFSLFL

SEQ ID NO: 63 - protein, 9 aa, synthetic construct
ALPGLVQRA

SEQ ID NO: 64 - protein, 9 aa, synthetic construct
FIFGLHLRL

SEQ ID NO: 65 - protein, 9 aa, synthetic construct
FITEKLPQV

SEQ ID NO: 66 - protein, 9 aa, synthetic construct
FLFSRWILL

SEQ ID NO: 67 - protein, 9 aa, synthetic construct
FLVKKKFFV

SEQ ID NO: 68 - protein, 9 aa, synthetic construct
GLCSLPPLL

SEQ ID NO: 69 - protein, 9 aa, synthetic construct
GLLRGMSRL

SEQ ID NO: 70 - protein, 9 aa, synthetic construct
LLHSLSTKV

SEQ ID NO: 71 - protein, 9 aa, synthetic construct
LLLERDPSL

SEQ ID NO: 72 - protein, 9 aa, synthetic construct
LLLGKCLGV

SEQ ID NO: 73 - protein, 9 aa, synthetic construct
RVTDVILFL

SEQ ID NO: 74 - protein, 9 aa, synthetic construct
SLAPDSRPV

SEQ ID NO: 75 - protein, 9 aa, synthetic construct
SLISVFNRA

SEQ ID NO: 76 - protein, 9 aa, synthetic construct
SLSHSVFPL

SEQ ID NO: 77 - protein, 9 aa, synthetic construct
SMLDHETFA

SEQ ID NO: 78 - protein, 9 aa, synthetic construct
SVDEKNFKM

SEQ ID NO: 79 - protein, 9 aa, synthetic construct
TLLSIPNYV

SEQ ID NO: 80 - protein, 9 aa, synthetic construct
TLSFFTLKL

SEQ ID NO: 81 - protein, 9 aa, synthetic construct
VQMEPTHFV

SEQ ID NO: 82 - protein, 9 aa, synthetic construct
YLSPFGHEI

SEQ ID NO: 83 - protein, 92 aa, synthetic construct
MRRHRPPPAATLRRNKKMRKKSMLFLWKRGRLLAKGQSLDHLLLEATANLLTAHWSSRGATSPHISTTTQRLPPLGRTILLPRGSSWTVSES

SEQ ID NO: 84 - protein, 9 aa, synthetic construct
RLPPLGRTI

SEQ ID NO: 85 - protein, 9 aa, synthetic construct
LPRGSSWTV

SEQ ID NO: 86 - protein, 9 aa, synthetic construct
LPPLGRTIL

SEQ ID NO: 87 - protein, 9 aa, synthetic construct
ATANLLTAH

SEQ ID NO: 88 - protein, 9 aa, synthetic construct
TQRLPPLGR

SEQ ID NO: 89 - protein, 9 aa, synthetic construct
ILLPRGSSW

SEQ ID NO: 90 - protein, 9 aa, synthetic construct
LLEATANLL

SEQ ID NO: 91 - protein, 9 aa, synthetic construct
KSMLFLWKR

SEQ ID NO: 92 - protein, 9 aa, synthetic construct
MLFLWKRGR

SEQ ID NO: 93 - protein, 9 aa, synthetic construct
KMRKKSMLF

SEQ ID NO: 94 - protein, 9 aa, synthetic construct
HRPPPAATL

SEQ ID NO: 95 - protein, 9 aa, synthetic construct
PPLGRTILL

SEQ ID NO: 96 - protein, 9 aa, synthetic construct
SPHISTTTQ

SEQ ID NO: 97 - protein, 9 aa, synthetic construct
GQSLDHLLL

SEQ ID NO: 98 - protein, 9 aa, synthetic construct
RPPPAATLR

(19) United States
(12) Patent Application Publication
(10) Pub. No.: US 2026/0102495 A1
(43) Pub. Date: Apr. 16, 2026

(54) METHOD FOR PRODUCING ONE OR SEVERAL SHARED CANCER EPITOPE(S) DERIVED FROM ALTERNATIVE TRANSLATIONAL CONTROL

(71) Applicants: CENTRE LEON BERARD, LYON (FR); ERVIMMUNE, LYON (FR); UNIVERSITE CLAUDE BERNARD LYON 1, VILLEURBANNE (FR); INSTITUT NATIONAL DE LA SANTE ET DE LA RECHERCHE MEDICALE (INSERM), PARIS (FR); CENTRE NATIONAL DE LA RECHERCHE SCIENTIFIQUE, PARIS (FR)

(72) Inventors: Stéphane DEPIL, Lyon (FR); Estelle BAULU, Lyon (FR); Nicolas CHUVIN, Lyon (FR); Jean-Jacques DIAZ, Lyon (FR); Virginie MARCEL, Lyon (FR); Nicole DALLA VENEZIA, Lyon (FR)

(73) Assignees: CENTRE LEON BERARD, LYON (FR); ERVIMMUNE, LYON (FR); UNIVERSITE CLAUDE BERNARD LYON 1, VILLEURBANNE (FR); INSTITUT NATIONAL DE LA SANTE ET DE LA RECHERCHE MEDICALE (INSERM), PARIS (FR); CENTRE NATIONAL DE LA RECHERCHE SCIENTIFIQUE, PARIS (FR)

(21) Appl. No.: 19/113,973

(22) PCT Filed: Sep. 22, 2023
(86) PCT No.: PCT/EP2023/076274

§ 371 Date: Mar. 21, 2025

(30) Foreign Application Priority Data
Sep. 23, 2022 (EP) 22306411.4

(51) Int. Cl. A61K 40/42; A61K 40/11; A61P 35/00; C07K 14/82; C07K 16/32; G01N 33/50; G16B 15/30

(52) U.S. Cl. CPC A61K 40/428; A61K 40/11; A61P 35/00; C07K 14/82; C07K 16/32; G01N 33/5091; G16B 15/30

(57) ABSTRACT

A method for producing or identifying one or several shared cancer epitope(s), as well as the peptides including or being made up of the epitopes identified or produced by the method, expression vectors encoding the peptides, cytotoxic T lymphocytes (CTLs) generated in vitro by stimulation of T cells with the peptides or vectors, CTLs of a subject treated with the peptides or vectors, and engineered T cells expressing T-cell receptors recognizing said peptides. Also, the use of the peptides, expression vectors, CTLs or engineered T cells as a vaccine or a medicament, and in particular, the use of the peptides, expression vectors, CTLs, or engineered T cells for preventing or treating at least one cancer in a subject in need thereof.

### FIELD OF INVENTION

The present invention relates to a method for producing or identifying one or several shared cancer epitope(s), as well as peptides comprising or consisting of the epitopes identified or produced by said method, expression vectors encoding said peptides, cytotoxic T lymphocytes (CTLs) generated in vitro by stimulation of T cells with the said peptides or vectors, CTLs of a subject treated with said peptides or vectors, and engineered T cells expressing T-cell receptors recognizing said peptides. The present invention also relates to the use of said peptides, expression vectors, CTLs or engineered T cells as a vaccine or a medicament, and in particular, the use of said peptides, expression vectors, CTLs, or engineered T cells for preventing or treating at least one cancer in a subject in need thereof.

### BACKGROUND OF INVENTION

The adaptive T cell immune response in cancer relies on the recognition of tumor epitopes specifically expressed by tumor cells. The role of neoantigens, generated by non-synonymous mutations specific to the tumor genome, has been extensively studied in the last decade and many clinical trials testing combinations of neoantigens in personalized cancer vaccines have been initiated, with encouraging preliminary results. However, determining the optimal combination of neoepitopes for each patient remains challenging. Furthermore, many tumors are characterized by a low or moderate tumor mutational burden. Therefore, unveiling other families of tumor antigens, such as those derived from alternative translation control, possibly shared among different patients with a same cancer or different cancer subtypes, is of utmost importance for the development of off-the-shelf therapies in tumors.

In the present application, the Applicants have developed a new method for identifying shared cancer epitopes, derived from alternative translation control. In particular, the Applicants have found new epitopes derived from c-myc gene that can induce specific T cell response, and have shown that the induced T cells are able to recognize and kill tumor cells.

### SUMMARY

The present invention relates to a method for producing one or several shared cancer epitope(s), wherein said method comprises the following steps:
(a) Identifying peptides derived from non-canonical initiation and/or termination of translation of a given gene, preferably an oncogene, and selecting among the identified peptides one or several shared cancer epitope(s),
(b) Generating the epitope(s) selected in step (a).

The present invention also relates to a method for identifying one or several shared cancer epitope(s), wherein said method comprises the following step:
(a) Identifying peptides derived from non-canonical initiation and/or termination of translation of a given gene, preferably an oncogene, and selecting among the identified peptides one or several shared cancer epitope(s).

In one embodiment, said step (a) comprises the following steps:
(a₁) Predicting the peptides derived from non-canonical initiation and/or termination of translation of a given gene,
(a₂) Identifying among the predicted peptides identified in step (a₁) the 8- to 15-mer peptides (i.e. epitopes) that bind to MHC class I molecules, preferably HLA-A molecules, more preferably HLA-A2 molecules,
(a₃) Identifying among the sequences of the 8- to 15-mer peptides (or epitopes) identified in step (a₂) the peptides (or epitopes) that are found in healthy subjects and/or healthy tissues and excluding said peptides (or epitopes), and
(a₄) Selecting among the remaining peptides (or epitopes) of step (a₃) one or several epitope(s) found in at least one cancer.

In one embodiment, the gene is an oncogene, preferably an oncogene selected from the group comprising or consisting of c-myc, and IGF1R, preferably the oncogene is c-myc.

In one embodiment, the cancer is a c-myc or an IGF1R associated cancer, preferably the cancer is breast cancer or colon cancer.

In one embodiment, said method further comprises an in vitro validation of the selected epitope(s) after step (a).

In one embodiment, said in vitro validation comprises at least one, preferably the three following steps:
(i) evaluating the induction of CD8+ T cell responses by the selected epitope(s),
(ii) evaluating the functionality of the CD8+ T cells specific for the selected epitope(s), and/or
(iii) evaluating the cytotoxicity of the CD8+ T cells specific for the selected epitope(s) in tumor cells and non-tumoral cells,
   optionally, wherein said in vitro validation further comprises a step (iv) of evaluating the expression of the selected epitope(s) in tumor cells, preferably wherein said expression is assessed by ribosome profiling or mass spectrometry.

The present invention further relates to a peptide comprising or consisting of an epitope identified or generated by the method as described hereinabove.

The present invention further relates to a peptide comprising or consisting of an epitope having a sequence selected in the group comprising or consisting of LLLEATANL (SEQ ID NO: 1), SLTDLYLRI (SEQ ID NO: 2), IMTASNWTL (SEQ ID NO: 3), AMSPQLHNI (SEQ ID NO: 4), GLAAPAPKL (SEQ ID NO: 5), GLPPHPAHL (SEQ ID NO: 6), GMPWPIPAV (SEQ ID NO: 7), SLQETSYAL (SEQ ID NO: 8), SLYPIACSL (SEQ ID NO: 9), SVLGHDFSV (SEQ ID NO: 10), VQDMIQTQV (SEQ ID NO: 11), ILDDWLRHL (SEQ ID NO: 12) and SLPSQHWSL (SEQ ID NO: 13), preferably the peptide comprises or consists of an epitope having a sequence selected in the group comprising or consisting of LLLEATANL (SEQ ID NO: 1), SLTDLYLRI (SEQ ID NO: 2) and IMTASNWTL (SEQ ID NO: 3).

The present invention further relates to a peptide comprising or consisting of an epitope having a sequence selected in the group comprising or consisting of LLLEATANL (SEQ ID NO: 1), SLTDLYLRI (SEQ ID NO: 2), AMSPQLHNI (SEQ ID NO: 4), GLAAPAPKL (SEQ ID NO: 5), GLPPHPAHL (SEQ ID NO: 6), GMPWPIPAV (SEQ ID NO: 7), SLQETSYAL (SEQ ID NO: 8), SLYPIACSL (SEQ ID NO: 9), SVLGHDFSV (SEQ ID NO: 10), VQDMIQTQV (SEQ ID NO: 11), ILDDWLRHL (SEQ ID NO: 12) and SLPSQHWSL (SEQ ID NO: 13), preferably the peptide comprises or consists of an epitope having a sequence selected in the group comprising or consisting of LLLEATANL (SEQ ID NO: 1) and SLTDLYLRI (SEQ ID NO: 2).

The present invention further relates to an expression vector inducing expression of one or more peptide(s) as described hereinabove.

The present invention further relates to a cytotoxic T-lymphocyte of a subject treated with one or more peptide(s) as described hereinabove, or one or more expression vector(s) as described hereinabove.

The present invention further relates to a cytotoxic T-lymphocyte generated in vitro by stimulation of T cells with one or more peptide(s) as described hereinabove, or one or more expression vector(s) as described hereinabove.

The present invention further relates to an engineered T cell expressing a T-cell receptor recognizing a peptide as described hereinabove.

The present invention further relates to one or more peptide(s) as described hereinabove, one or more expression vector(s) as described hereinabove, one or more cytotoxic T-lymphocyte(s) as described hereinabove, or one or more engineered T cell(s) as described hereinabove for use as a vaccine or medicament.

The present invention further relates to one or more peptide(s) as described hereinabove, one or more expression vector(s) as described hereinabove, one or more cytotoxic T-lymphocyte(s) as described hereinabove, or one or more engineered T cell(s) as described hereinabove for use in treating or preventing at least one cancer in a subject in need thereof.

### Definitions

In the present invention, the following terms have the following meanings:

“C-myc” refers to the proto-oncogene myc coding for a nuclear protein which is involved in nucleic acid metabolism and in mediating the cellular response to growth factors. Truncation of the first exon, which appears to regulate c-myc expression, is crucial for tumorigenicity. The human c-myc gene is located at 8q24 on the long arm of chromosome 8.

“Epitope” refers to a portion of an antigen, that is capable of stimulating an immune response.

“Shared cancer epitope” refers to an epitope that is not specific to a given subject. A shared cancer epitope may be shared between different patients with the same cancer histology or between different patients with different cancer histologies.

“Frameshift” refers to a change in the open reading frame by one or more bases in either the 5′ or 3′ directions during translation.

“Insulin-like growth factor 1 receptor” or “IGFR1” refers to a gene on chromosome 15q26.3 that encodes a tyrosine kinase receptor with a high binding affinity for insulin-like growth factor, which plays a key role in transformation events in cell growth and survival.

“Internal ribosome entry site” or “IRES” are sequences that can recruit ribosomes and allow translation.

“Mass spectrometry” refers to an analytical method used in determining the identity of a chemical based on its mass using mass analyzers/mass spectrometers.

“Non-canonical initiation and/or termination of translation of a gene” refers to non-conventional mechanisms of translation initiation and/or termination, which may be induced under stress conditions such as, for example, hypoxia, apoptosis, starvation, and viral infection. The conventional mechanism for translation initiation involves recruitment of the 40S ribosome to the cap structure at the 5′ end of the mRNA, followed by linear scanning of the 5′-UTR until the initiation codon is reached. The non-canonical mechanisms of initiation and/or termination of translation of a gene includes, without limitation, the following events: frameshifts, readthroughs, translation of regions on 5′UTR and/or 3′UTR that are normally non translated (e.g. initiation of translation upstream of the initiation codon, termination of translation downstream of the stop codon, and/or initiation of translation occurring downstream of the stop codon of the coding sequence), and IRES-dependent initiation of translation.

“Oncogenes” refers to genes whose gain-of-function alterations lead to neoplastic cell transformation. They include, for example, genes for activators or stimulators of cell proliferation such as growth factors, growth factor receptors, protein kinases, signal transducers, nuclear phosphoproteins, and transcription factors.

“Open reading frame” or “ORF” refers to a sequence of nucleotide triplets that code for amino acids located between an initiation codon and a stop codon in the same reading frame.

“Peptide” refers to a linear polymer of amino acids of at least 2, 3, 4, 5, 6, 7, 8, 9, 10, 11, 12, 13, 14, 15 amino acids linked together by peptide bonds. Amino acid residues in peptides are abbreviated as follows: Phenylalanine is Phe or F; Leucine is Leu or L; Isoleucine is Ile or I; Methionine is Met or M; Valine is Val or V; Serine is Ser or S; Proline is Pro or P; Threonine is Thr or T; Alanine is Ala or A; Tyrosine is Tyr or Y; Histidine is His or H; Glutamine is Gln or Q; Asparagine is Asn or N; Lysine is Lys or K; Aspartic Acid is Asp or D; Glutamic Acid is Glu or E; Cysteine is Cys or C; Tryptophan is Trp or W; Arginine is Arg or R; and Glycine is Gly or G. Said peptides may comprise non-standard amino acids, which refer to those amino acids that have been chemically modified after they have been incorporated into a protein (called a “posttranslational modification”) and those amino acids that occur in living organisms but are not found in proteins. Posttranslational modifications include, for example, phosphorylation and glycosylation of amino acids. Examples of such non-standard amino acids include, without limitation, selenocysteine, cystine, desmosine, isodesmosine, hydroxyproline and hydroxylysine, gamma-carboxyglutamate, phosphoserine, phosphothreonine, phosphotyrosine, and inositol.

“Prevent”, “preventing” and “prevention” refer to prophylactic and preventative measures, wherein the object is to reduce the chances that a subject will develop the pathologic condition or disorder over a given period of time. Such a reduction may be reflected, e.g., in a delayed onset of at least one symptom of the pathologic condition or disorder in the subject.

“Readthrough” refers to a process wherein, in translation, a stop codon is interpreted as a sense codon.

“Ribosome profiling” or “Ribo-seq” refers to a method based on deep sequencing of ribosome-protected mRNA fragments (also called “footprints”). The ribosome footprints typically show precise positioning between the start and stop codon of a gene, which enables global and experimental genomic coding region identification. It also enables to show precise positioning of the ribosome on the mRNA.

“Subject” refers to a mammal, preferably a human. In one embodiment, a subject may be a “patient”, i.e., a warm-blooded animal, more preferably a human, who/which is awaiting the receipt of, or is receiving medical care or was/is/will be the object of a medical procedure, or is monitored for the development of a disease. The term “mammal” refers here to any mammal, including humans, domestic and farm animals, and zoo, sports, or pet animals, such as dogs, cats, cattle, horses, sheep, pigs, goats, rabbits, etc. Preferably, the mammal is a primate, more preferably a human.

“Therapeutically effective amount” refers to the level or amount of one or more peptide(s), one or more expression vector(s), one or more CTL(s), or one or more engineered T cell(s) herein that is aimed at, without causing significant negative or adverse side effects to the target, (1) delaying or preventing the onset of a disease, disorder, or condition; (2) slowing down or stopping the progression, aggravation, or deterioration of one or more symptoms of the disease, disorder, or condition; (3) bringing about ameliorations of the symptoms of the disease, disorder, or condition; (4) reducing the severity or incidence of the disease, disorder, or condition; or (5) curing the disease, disorder, or condition. A therapeutically effective amount may be administered prior to the onset of the disease, disorder, or condition, for a prophylactic or preventive action. Alternatively, or additionally, the therapeutically effective amount may be administered after initiation of the disease, disorder, or condition, for a therapeutic action.

“Treating” or “treatment” or “alleviation” refers to therapeutic treatment; wherein the object is to slow down (lessen) the targeted pathologic condition or disorder. A subject or mammal is successfully “treated” for a cancer if, after receiving a therapeutic amount of the one or more peptide(s), one or more expression vector(s), one or more cytotoxic T lymphocyte(s) or one or more engineered T cell(s) according to the present invention, the patient shows observable and/or measurable reduction in or absence of one or more of the following: reduction in the number of cancer cells (or tumor size); reduction in the percent of total cells that are cancerous; and/or relief to some extent of one or more of the symptoms associated with the specific disease or condition; reduced morbidity and mortality, and improvement in quality of life issues. The above parameters for assessing successful treatment and improvement in the disease are readily measurable by routine procedures familiar to a physician.

“Vaccine” refers to a compound that, once administered to a subject, may induce a humoral and/or cellular immune response, and this immune response is protective.

“Vector”, or “expression vector” means the vehicle by which a DNA or RNA sequence (e.g. a foreign gene) can be introduced into a host cell, so as to transform the host and promote expression (e.g. transcription and translation) of the introduced sequence.

“uORF” refer to short coding sequences flanked by a start codon and a stop codon upstream of the main open reading frame (mORF).

### DETAILED DESCRIPTION

This invention relates to a method for producing one or several shared cancer epitope(s), wherein said method comprises the following steps:
(a) Identifying peptides derived from non-canonical initiation and/or termination of translation of a given gene, preferably an oncogene, and selecting among the identified peptides one or several shared cancer epitope(s), and
(b) Generating the epitope(s) selected in step (a).

This invention also relates to a method for identifying one or several shared cancer epitope(s), wherein said method comprises the following step:
(a) Identifying peptides derived from non-canonical initiation and/or termination of translation of a given gene, preferably an oncogene, and selecting among the identified peptides one or several shared cancer epitope(s).

In one embodiment, the step (a) is done with an in silico approach.

In one embodiment, the step (a) comprises the step (a₁) of predicting the peptides derived from non-canonical initiation and/or termination of translation of a given gene.

In one embodiment, the step (a₁) comprises predicting the peptides derived from at least one mechanism of non-canonical initiation and/or termination of translation of a given gene.

Examples of mechanisms of non-canonical initiation and/or termination of translation of a given gene include, without limitation, frameshifts of the open reading frame, stop codon readthroughs, translation of 5′UTR and/or 3′UTR, or IRES-dependent initiation of translation.

By “translation of 5′UTR and/or 3′UTR” or “translation of 3′ and/or 5′ regions”, it is meant that the 5′UTR and/or the 3′UTR of a given mRNA that are normally non-translated in a canonical translation, are translated. Translation of 5′UTR means that the initiation of translation starts upstream of the initiation codon. Translation of 3′UTR means that the termination of translation stops downstream of the stop codon and/or that initiation of translation occurs downstream of the stop codon of the coding sequence. The translation of the 5′UTR and/or 3′UTR may be a complete or a partial translation of the 5′UTR and/or 3′UTR.

In one embodiment, the at least one mechanism of non-canonical initiation and/or termination of translation of a given gene is selected among:
a frameshift of the open reading frame,
a stop codon readthrough,
a translation of 5′UTR and/or 3′UTR,
IRES-dependent initiation of translation, and/or
upstream ORF (uORF).

In one embodiment, the at least one mechanism of non-canonical initiation and/or termination of translation of a given gene is selected among:
a frameshift of the open reading frame,
a stop codon readthrough,
a translation of 5′UTR and/or 3′UTR, and/or
an IRES-dependent initiation of translation.

In one embodiment, the step (a₁) comprises predicting the peptides derived from one, two, or three mechanism(s) of non-canonical initiation and/or termination of translation of a given gene.

In one embodiment, the one, two, or three mechanism(s) of non-canonical initiation and/or termination of translation of a given gene are selected among:
a frameshift of the open reading frame,
a stop codon readthrough,
a translation of 5′UTR and/or 3′UTR, and/or
an IRES-dependent initiation of translation.

In one embodiment, the step (a₁) comprises predicting the peptides derived from the four mechanisms of non-canonical initiation and/or termination of translation of a given gene:
a frameshift of the open reading frame,
a stop codon readthrough,
a translation of 5′UTR and/or 3′UTR, and
an IRES-dependent initiation of translation.

In one embodiment, the step (a₁) comprises predicting the peptides derived from one, two, three or four mechanism(s) of non-canonical initiation and/or termination of translation of a given gene selected among:
a frameshift of the open reading frame,
a stop codon readthrough,
a translation of 5′UTR and/or 3′UTR, and
an IRES-dependent initiation of translation, and/or
upstream ORF (uORF).

In one embodiment, the step (a) comprises the step (a₂) of identifying among the predicted peptides identified in step (a₁) the 8- to 15-mer peptides that bind to MHC class I molecules. As used, the step (a₂) enables to select, among the peptides identified in step (a₁), 8- to 15-mer peptides that bind to MHC class I molecules, i.e. epitopes binding to MHC class I molecules.

Analysis tools for predicting the binding of sequences (i.e. epitopes) with MHC molecules are well known by the skilled artisan in the art and include, for example, MHCflurry (T. J. O'Donnell et al., MHCflurry: Open-Source Class I MHC Binding Affinity Prediction. Cell Systems. 7, 129-132.e4 (2018)) or NetMHCPan (Reynisson et al., NetMHCpan-4.1 and NetMHCIIpan-4.0: improved predictions of MHC antigen presentation by concurrent motif deconvolution and integration of MS MHC eluted ligand data, Nucleic Acids Res, 2020 Jul. 2; 48 (W1): W449-W454).

In one embodiment, said MCH class I molecule is a HLA molecule. In one embodiment, the MCH class I molecule is selected from the group comprising or consisting of HLA-A, HLA-B and HLA-C molecules. In one embodiment, the MCH class I molecule is a HLA-A molecule, such as an HLA-A2 molecule.

In one embodiment, the 8- to 15-mer peptides (or epitopes) identified in step (a₂) strongly bind to MHC class I molecules, preferably HLA-A2 molecules.

In one embodiment, the strong binder peptides (or epitopes) are selected using a percentile rank equal or inferior to 0.5%, which is based on the likelihood of this peptide being presented when compared to a pool of natural ligands.

In one embodiment, the peptides (or epitopes) identified in step (a₂) are 8- to 15-mer peptides, i.e. peptides comprising from 8 to 15 amino acids.

In one embodiment, the peptides (or epitopes) identified in step (a₂) are 9- to 10-mer peptides, i.e. peptides comprising from 9 to 10 amino acids.

In one embodiment, the peptides (or epitopes) identified in step (a₂) are 8-, 9-, 10-, 11-, 12-, 13-, 14-, or 15-mer peptides, i.e. peptides respectively comprising 8, 9, 10, 11, 12, 13, 14, or 15 amino acids. In one embodiment, the peptides (or epitopes) identified in step (a₂) are 9-mer peptides.

In one embodiment, the step (a) comprises the step (a₃) of identifying among the sequences of the 8- to 15-mer peptides (or epitopes) identified in step (a₂) the peptides (or epitopes) that are found in healthy subjects (i.e. subjects not affected with a cancer) and excluding said peptides. This step notably enables to confirm that the selected peptides (or epitopes) do not match any self-protein of subjects not affected with cancer.

In one embodiment, the step (a₃) comprises the step of aligning the sequences of the 8- to 15-mer peptides (or epitopes) identified in step (a₂) with the normal human proteome (i.e. human proteome from healthy subjects) and excluding those with a perfect sequence homology to the normal human proteome. Alignment may be done with BLAST protein database, or refseq_protein database.

In one embodiment, the step (a₃) comprises the step of comparing the sequences of the 8- to 15-mer peptides (or epitopes) identified in step (a₂) with normal tissue proteomic database (i.e. tissue proteomic database from healthy tissues) and excluding those found in healthy/normal tissues. Data of normal tissue proteomic database can be found in public database, such as, for example, in Genotype-Tissue Expression (GTEx) database.

As used herein, healthy or normal tissues refer to tissues not affected with cancer.

In one embodiment, the step (a₃) comprises at least one of the steps described hereinabove (i.e. the alignment with the human proteome or the comparison with tissue proteomic database).

In one embodiment, the step (a₃) comprises the two steps described hereinabove. Thus, in one embodiment, the step (a₃) comprises the following steps:
aligning the sequences of the 8- to 15-mer peptides (or epitopes) identified in step (a₂) with the normal human proteome and excluding those with a perfect sequence homology to the normal human proteome, and
comparing the sequences of the remaining peptides (or epitopes) with normal tissue proteomic database and excluding those found in healthy/normal tissues.

In one embodiment, the step (a) comprises the step (a₄) of selecting among the remaining peptides (or epitopes) of step (a₃), one or several epitope(s) found in at least one cancer. Step (a₄) thus allows, for example, to select one or several epitope(s) that is/are commonly shared in different patients with a same cancer or in different patients with different cancers.

In one embodiment, the step (a₄) comprises selecting among the remaining peptides (or epitopes) of step (a₃), one or several epitope(s) found in proteomic mass spectrometric database of at least one cancer.

Examples of proteomic mass spectrometric databases of cancers include, without limitation, include data from The Cancer Genome Atlas (TCGA) and data from Clinical Proteomic Tumor Analysis Consortium (CPTAC).

In one embodiment, the step (a₄) comprises selecting among the remaining peptides (or epitopes) of step (a₃), one or several epitope(s) found in at least one, at least two, or more cancers.

In one embodiment, the at least one cancer is a c-myc associated cancer. In one embodiment, the at least one cancer is an IGF1R associated cancer.

As used herein, c-myc associated cancers are cancers in which a deregulation of c-myc expression occurs. As used herein, IGF1R associated cancers are cancers in which a deregulation of IGF1R expression occurs.

Examples of c-myc associated cancers include, without limitation, colon, breast, lung, prostate, bladder cancers and lymphomas. Examples of IGF1R associated cancers include, without limitation, breast, colon, lung, prostate cancer, and sarcomas.

In one embodiment, the at least one cancer is selected from the group comprising or consisting of breast cancer, including triple negative breast cancer, ovarian cancer, melanoma, sarcoma, teratocarcinoma, colon cancer, prostate cancer, bladder cancer, lung cancer, including non-small cell lung carcinoma and small cell lung carcinoma, head and neck cancer, colorectal cancer, glioblastoma, leukemias, lymphomas and other solid tumors and hematological malignancies.

In one embodiment, the at least one cancer is colon cancer. In one embodiment, the at least one cancer is breast cancer.

In one embodiment, the step (a₄) comprises selecting among the remaining peptides (or epitopes) of step (a₃), one or several epitope(s) found in at least one cancer, wherein the cancer is breast or colon cancer.

In one embodiment, the step (a₄) comprises selecting among the remaining peptides (or epitopes) of step (a₃), one or several epitope(s) found in breast and colon cancers.

In one embodiment, the shared cancer epitope(s) is/are epitope(s) found in at least one cancer mentioned hereinabove. In one embodiment, the shared cancer epitope(s) is/are epitope(s) found in breast and/or colon cancer(s).

In one embodiment, the step (a) comprises at least one, preferably the four steps (a₁), (a₂), (a₃), and (a₄), as mentioned hereinabove.

In one embodiment, the step (a) comprises the following steps:
(a₁) Predicting the peptides derived from non-canonical initiation and/or termination of translation of a given gene,
(a₂) Identifying among the predicted peptides identified in step (a₁) the 8- to 15-mer peptides (i.e. epitopes) that bind to MHC class I molecules, preferably HLA-A molecules, more preferably HLA-A2 molecules,
(a₃) Identifying among the sequences of the 8- to 15-mer peptides (or epitopes) identified in step (a₂) the peptides (or epitopes) that are found in healthy subjects (i.e. subjects not affected with a cancer) and/or healthy tissues and excluding said peptides (or epitopes), and
(a₄) Selecting among the remaining peptides (or epitopes) of step (a₃) one or several epitope(s) found in at least one cancer.

In one embodiment, the step (a) comprises the following steps:
(a₁) Predicting the peptides derived from non-canonical initiation and/or termination of translation of a given gene,
(a₂) Identifying among the predicted peptides identified in step (a₁) the 8- to 15-mer peptides (i.e. epitopes) that bind to MHC class I molecules, preferably HLA-A molecules, more preferably HLA-A2 molecules,
(a₃) Identifying among the sequences of the 8- to 15-mer peptides (or epitopes) identified in step (a₂) the peptides (or epitopes) that are found in healthy subjects and/or healthy tissues and excluding said peptides (or epitopes), with the following steps:
aligning the sequences of the 8- to 15-mer peptides (or epitopes) identified in step (a₂) with the normal human proteome and excluding those with a perfect sequence homology to the normal human proteome, and
comparing the sequences of the remaining peptides (or epitopes) with normal tissue proteomic database and excluding those found in healthy/normal tissues, and
(a₄) Selecting among the remaining peptides (or epitopes) of step (a₃) one or several epitope(s) found in at least one cancer.

In one embodiment, the given gene is an oncogene.

In one embodiment, the given gene is an IRES (internal ribosome entry site)-dependent gene. Examples of IRES-dependent gene include, without limitation, c-myc and IGF1R.

In one embodiment, the gene is an oncogene, preferably selected from the group comprising or consisting of c-myc and IGF1R.

In one embodiment, the gene is c-myc. In one embodiment, the gene is IGF1R.

In one embodiment, the step (b) comprises the step of producing the epitope(s) identified in step (a). Said epitopes may be made by any technique known to those of skill in the art, including the expression of proteins, polypeptides or peptides through standard molecular biological techniques such as; for example, recombinant approaches, the isolation of proteins or peptides from natural sources, or the chemical synthesis of proteins or peptides.

As used herein, chemic synthesis relates to the synthesis of the epitope(s) identified in step (a) by adding amino acids and/or fragments of the epitope(s) successively to the mixture, to react with the growing chain. Said chemical synthesis may be performed in liquid- or solid-phase.

As used herein, recombinant approaches relate to the synthesis of the epitope(s) identified in step (a) by expressing an expression vector encoding the epitope(s) in a host cell, and collecting the epitopes produced by the host cell.

In one embodiment, a method to obtain the epitope(s) as described hereinabove comprises:
introducing in vitro or ex vivo a vector encoding the epitope into a competent host cell;
culturing in vitro or ex vivo host cells transformed with the expression vector, under conditions suitable for expression of the epitope(s);
optionally, selecting the cells which express and/or secrete said epitope(s); and
recovering the expressed epitope(s).

In one embodiment, the method as described hereinabove further comprises a step of in vitro validation of the selected epitope(s) after step (a).

In one embodiment, the method described hereinabove is combined with one or several step(s) described hereinbelow. In one embodiment, the method described hereinabove is combined with one, two, three or four step(s) described hereinbelow.

In one embodiment, the in vitro validation comprises the step (i) of evaluating the induction of T cell responses, such as, for example, CD8+ T cell responses, by the selected epitope(s).

In one embodiment, the induction of T cells responses is assessed by measuring the induction of T cells, such as, for example, CD8+ T cells, specific for the selected epitopes.

Examples of methods to induce CD8+ T cells include, for example, in vitro or ex vivo priming assays with the selected epitopes. As an example, human monocyte-derived dendritic cells may be pulsed with the selected epitope(s) to induce specific CD8+ T cells.

Examples of methods to assess the induction of CD8+ T cells include, for example, a dextramer-based quantification.

In one embodiment, the in vitro validation comprises the step (ii) of evaluating the functionality of T cells, such as, for example, CD8+ T cells or TCR-engineered T cells, specific for the selected epitope(s). As used herein, TCR-engineered T cells refer to engineered T cells expressing a TCR recognizing the selected epitope(s).

In one embodiment, the functionality of the T cells, such as, for example, CD8+ T cells or TCR-engineered T cells, specific for the selected epitope(s) is assessed by measuring production of IFN-γ, TNFα or granzyme B in presence of epitopes-stimulated cells. Examples of methods for measuring production of such molecules include, for example, flow cytometry, ELISA or fluorospot assays.

In one embodiment, the functionality of the T cells, such as, for example, CD8+ T cells or TCR-engineered T cells, specific for the selected epitope(s) is assessed by measuring the extracellular staining of markers, such as, 4-1BB (CD137) or CD107a.

In one embodiment, the in vitro validation comprises the step (iii) of evaluating the cytotoxicity of T cells, such as, for example, CD8+ T cells or TCR-engineered T cells, specific for the selected epitope(s) in tumor cells and non-tumoral cells.

As used herein, the expressions “cell”, “cell line,” and “cell culture” are used interchangeably.

In one embodiment, the cytotoxicity of T cells specific for the selected epitope(s) is assessed by measuring the cell death of cells presenting the cognate epitopes at the cell surface in presence of epitope-specific T cells, such as, for example, CD8+ T cells or TCR-engineered T cells.

In one embodiment, the cells do not naturally express the selected epitope(s) and may be pulsed with the cognate epitopes. As an example, T2 cells pulsed with the selected epitope(s) may be co-cultured with CD8+ T cells or TCR-engineered T cells specific for the selected epitope(s) and the death of T2 cells may be measured.

As used herein, “cells that do not naturally express the selected epitope(s)” means that the cells do not naturally comprise the molecular machinery necessary for the expression of the selected epitope(s) and for its expression at the cell surface.

In one embodiment, the cells naturally express the selected epitope(s). As an example, tumor cell lines, such as MDA-MB-231 or HCT116, may be co-cultured with CD8+ T cells or TCR-engineered T cells specific for the selected epitope(s) and the death of tumor cells may be measured.

As used herein, “cells that naturally express the selected epitope(s)” means that the cells naturally comprise all the molecular machinery necessary for the expression of the selected epitope(s) and for its expression at the cell surface.

In one embodiment, the in vitro validation comprises the step (iv) of evaluating the expression of the selected epitope(s) in tumor cells.

In one embodiment, the expression of the selected epitope(s) in tumor cells is assessed by ribosome profiling (or ribo-seq).

In one embodiment, the expression of the selected epitope(s) in tumor cells is assessed by mass spectrometry.

In one embodiment, the expression of the selected epitope(s) in tumor cells is assessed by a valid-NEO method. As used herein, valid-NEO is a multi-omics platform for neoantigen detection and quantification from limited clinical samples.

In one embodiment, the in vitro validation comprises at least one, preferably the three following steps:
(i) evaluating the induction of CD8+ T cell responses by the selected epitope(s),
(ii) evaluating the functionality of T cells, such as, for example CD8+ T cells or TCR-engineered T cells, specific for the selected epitope(s), and/or
(iii) evaluating the cytotoxicity of T cells, such as, for example CD8+ T cells or TCR-engineered T cells, specific for the selected epitope(s) in tumor cells and non-tumoral cells,
   optionally, wherein said in vitro validation further comprises a step (iv) of evaluating the expression of the selected epitope(s) in tumor cells, preferably wherein said expression is assessed by ribosome profiling or mass spectrometry.

As compared to the methods of the prior art, the method as described herein may present one or several of the following advantage(s). In some embodiments, the method as described herein enables to detect cancer epitope(s) with a higher sensibility, as compared to the methods of the prior art. In some embodiments, the method as described herein enables to identify cancer epitope(s) that are weakly expressed in tumors, and that may not be identified by methods of the prior art. In some embodiments, the method as described herein enables, especially when applied to oncogenes, to detect cancer epitope(s) that is/are shared, i.e. that is/are shared by different subjects suffering from a same type of cancer or by different subjects suffering from different types of cancers. In some embodiments, the method as described herein enables, especially when applied to oncogenes, to detect cancer epitope(s) that has/have a low risk of escape by deletion or mutation (i.e. the cancer epitope(s) has/have a low risk of being deleted or mutated during a patient's life).

This invention further relates to a peptide comprising or consisting of an epitope identified or generated by the method as described hereinabove.

The present invention relates to a peptide comprising or consisting of an epitope having a sequence selected in the group comprising or consisting of: LLLEATANL (SEQ ID NO: 1), SLTDLYLRI (SEQ ID NO: 2), IMTASNWTL (SEQ ID NO: 3), AMSPQLHNI (SEQ ID NO: 4), GLAAPAPKL (SEQ ID NO: 5), GLPPHPAHL (SEQ ID NO: 6), GMPWPIPAV (SEQ ID NO: 7), SLQETSYAL (SEQ ID NO: 8), SLYPIACSL (SEQ ID NO: 9), SVLGHDFSV (SEQ ID NO: 10), VQDMIQTQV (SEQ ID NO: 11), ILDDWLRHL (SEQ ID NO: 12), SLPSQHWSL (SEQ ID NO: 13), FLLMPLSFL (SEQ ID NO: 14), IILGIVFLL (SEQ ID NO: 15), SLDHLLLEA (SEQ ID NO: 16), FLWKRGRLL (SEQ ID NO: 17), ALLDGVLPA (SEQ ID NO: 18), LLFKVDFFL (SEQ ID NO: 19), RLGAAVFLL (SEQ ID NO: 20), RLLAKGQSL (SEQ ID NO: 21), SQPPPSLFV (SEQ ID NO: 22), ALLRIGHTL (SEQ ID NO: 23), GQASVPLFL (SEQ ID NO: 24), KLQTLLASI (SEQ ID NO: 25), LLASILFYI (SEQ ID NO: 26), LLVSTGVTV (SEQ ID NO: 27), QMQPHNLGV (SEQ ID NO: 28), SLFKLQTLL (SEQ ID NO: 29), VMFFKSQHL (SEQ ID NO: 30), ALADEWRNL (SEQ ID NO: 31), ALMISLGSV (SEQ ID NO: 32), ALWQDHTEI (SEQ ID NO: 33), AQWPAPRLV (SEQ ID NO: 34), CLLSKPVRL (SEQ ID NO: 35), FLFSICKQL (SEQ ID NO: 36), FLLTPRNFL (SEQ ID NO: 37), FMMITAYTV (SEQ ID NO: 38), FSIELLFSV (SEQ ID NO: 39), GLFFSLMFL (SEQ ID NO: 40), GLKPWTQYA (SEQ ID NO: 41), KLFGFCFQL (SEQ ID NO: 42), KLISELRRI (SEQ ID NO: 43), KLSELLMSF (SEQ ID NO: 44), LLFSVNREV (SEQ ID NO: 45), LLLAGGPGL (SEQ ID NO: 46), LLPGGLLLL (SEQ ID NO: 47), LLPPAPLVV (SEQ ID NO: 48), LLQALMISL (SEQ ID NO: 49), LLVISLWSV (SEQ ID NO: 50), LLWKLISEL (SEQ ID NO: 51), QIIQLVIRV (SEQ ID NO: 52), RLAPLFQQL (SEQ ID NO: 53), SLKDGVFTT (SEQ ID NO: 54), SLSWETPGV (SEQ ID NO: 55), SLWPHPTTV (SEQ ID NO: 56), SMMGRMPAA (SEQ ID NO: 57), SVHPTAPAV (SEQ ID NO: 58), SVPKHVWEA (SEQ ID NO: 59), VLFKLSELL (SEQ ID NO: 60), VLFSILVST (SEQ ID NO: 61), ALLTFSLFL (SEQ ID NO: 62), ALPGLVQRA (SEQ ID NO: 63), FIFGLHLRL (SEQ ID NO: 64), FITEKLPQV (SEQ ID NO: 65), FLFSRWILL (SEQ ID NO: 66), FLVKKKFFV (SEQ ID NO: 67), GLCSLPPLL (SEQ ID NO: 68), GLLRGMSRL (SEQ ID NO: 69), LLHSLSTKV (SEQ ID NO: 70), LLLERDPSL (SEQ ID NO: 71), LLLGKCLGV (SEQ ID NO: 72), RVTDVILFL (SEQ ID NO: 73), SLAPDSRPV (SEQ ID NO: 74), SLISVENRA (SEQ ID NO: 75), SLSHSVFPL (SEQ ID NO: 76), SMLDHETFA (SEQ ID NO: 77), SVDEKNFKM (SEQ ID NO: 78), TLLSIPNYV (SEQ ID NO: 79), TLSFFTLKL (SEQ ID NO: 80), VQMEPTHFV (SEQ ID NO: 81), YLSPFGHEI (SEQ ID NO: 82), RLPPLGRTI (SEQ ID NO: 84), LPRGSSWTV (SEQ ID NO: 85), LPPLGRTIL (SEQ ID NO: 86), ATANLLTAH (SEQ ID NO: 87), TQRLPPLGR (SEQ ID NO: 88), ILLPRGSSW (SEQ ID NO: 89), LLEATANLL (SEQ ID NO: 90), KSMLFLWKR (SEQ ID NO: 91), MLFLWKRGR (SEQ ID NO: 92), KMRKKSMLF (SEQ ID NO: 93), HRPPPAATL (SEQ ID NO: 94), PPLGRTILL (SEQ ID NO: 95), SPHISTTTQ (SEQ ID NO: 96), GQSLDHLLL (SEQ ID NO: 97) and RPPPAATLR (SEQ ID NO: 98).

The present invention relates to a peptide comprising or consisting of an epitope having a sequence selected in the group comprising or consisting of: FLLMPLSFL (SEQ ID NO: 14), IILGIVFLL (SEQ ID NO: 15), LLLEATANL (SEQ ID NO: 1), SLDHLLLEA (SEQ ID NO: 16), SLTDLYLRI (SEQ ID NO: 2), FLWKRGRLL (SEQ ID NO: 17), ALLDGVLPA (SEQ ID NO: 18), LLFKVDFFL (SEQ ID NO: 19), RLGAAVFLL (SEQ ID NO: 20), RLLAKGQSL (SEQ ID NO: 21), SQPPPSLFV (SEQ ID NO: 22), ALLRCGHTL (SEQ ID NO: 23), GQASVPLFL (SEQ ID NO: 24), KLQTLLASI (SEQ ID NO: 25), LLASILFYI (SEQ ID NO: 26), LLVSTGVTV (SEQ ID NO: 27), and VMFFKSQHL (SEQ ID NO: 30),

The present invention relates to a peptide comprising or consisting of an epitope having a sequence selected in the group comprising or consisting of: ALADEWRNL (SEQ ID NO: 31), ALMISLGSV (SEQ ID NO: 32), ALWQDHTEI (SEQ ID NO: 33), AMSPQLHNI (SEQ ID NO: 4), AQWPAPRLV (SEQ ID NO: 34), CLLSKPVRL (SEQ ID NO: 35), FLFSICKQL (SEQ ID NO: 36), FLLTPRNFL (SEQ ID NO: 37), FMMITAYTV (SEQ ID NO: 38), FSIELLFSV (SEQ ID NO: 39), GLFFSLMFL (SEQ ID NO: 40), GLKPWTQYA (SEQ ID NO: 41), KLFGFCFQL (SEQ ID NO: 42), KLISELRRI (SEQ ID NO: 43), KLSELLMSF (SEQ ID NO: 44), LLFSVNREV (SEQ ID NO: 45), LLLAGGPGL (SEQ ID NO: 46), LLPGGLLLL (SEQ ID NO: 47), LLPPAPLVV (SEQ ID NO: 48), LLQALMISL (SEQ ID NO: 49), LLVISLWSV (SEQ ID NO: 50), LLWKLISEL (SEQ ID NO: 51), QIIQLVIRV (SEQ ID NO: 52), RLAPLFQQL (SEQ ID NO: 53), SLKDGVFTT (SEQ ID NO: 54), SLSWETPGV (SEQ ID NO: 55), SLWPHPTTV (SEQ ID NO: 56), SMMGRMPAA (SEQ ID NO: 57), SVHPTAPAV (SEQ ID NO: 58), SVPKHVWEA (SEQ ID NO: 59), VLFKLSELL (SEQ ID NO: 60), VLFSILVST (SEQ ID NO: 61), ALLTFSLFL (SEQ ID NO: 62), ALPGLVQRA (SEQ ID NO: 63), FIFGLHLRL (SEQ ID NO: 64), FITEKLPQV (SEQ ID NO: 65), FLFSRWILL (SEQ ID NO: 66), FLVKKKFFV (SEQ ID NO: 67), GLCSLPPLL (SEQ ID NO: 68), GLLRGMSRL (SEQ ID NO: 69), LLHSLSTKV (SEQ ID NO: 70), LLLERDPSL (SEQ ID NO: 71), LLLGKCLGV (SEQ ID NO: 72), RVTDVILFL (SEQ ID NO: 73), SLAPDSRPV (SEQ ID NO: 74), SLISVENRA (SEQ ID NO: 75), SLSHSVFPL (SEQ ID NO: 76), SMLDHETFA (SEQ ID NO: 77), SVDEKNFKM (SEQ ID NO: 78), TLLSIPNYV (SEQ ID NO: 79), TLSFFTLKL (SEQ ID NO: 80), VQMEPTHFV (SEQ ID NO: 81), and YLSPFGHEI (SEQ ID NO: 82).

The present invention relates to a peptide comprising or consisting of an epitope having a sequence selected in the group comprising or consisting of: LLLEATANL (SEQ ID NO: 1), SLDHLLLEA (SEQ ID NO: 16), RLLAKGQSL (SEQ ID NO: 21), FLWKRGRLL (SEQ ID NO: 17), RLPPLGRTI (SEQ ID NO: 84), LPRGSSWTV (SEQ ID NO: 85), LPPLGRTIL (SEQ ID NO: 86), ATANLLTAH (SEQ ID NO: 87), TQRLPPLGR (SEQ ID NO: 88), and ILLPRGSSW (SEQ ID NO: 89).

The present invention relates to a peptide comprising or consisting of an epitope having a sequence selected in the group comprising or consisting of: LLEATANLL (SEQ ID NO: 90), KSMLFLWKR (SEQ ID NO: 91), MLFLWKRGR (SEQ ID NO: 92), KMRKKSMLF (SEQ ID NO: 93), HRPPPAATL (SEQ ID NO: 94), PPLGRTILL (SEQ ID NO: 95), SPHISTTTQ (SEQ ID NO: 96), GQSLDHLLL (SEQ ID NO: 97) and RPPPAATLR (SEQ ID NO: 98).

The present invention relates to a peptide comprising or consisting of an epitope having a sequence selected in the group comprising or consisting of: LLLEATANL (SEQ ID NO: 1), SLTDLYLRI (SEQ ID NO: 2), IMTASNWTL (SEQ ID NO: 3), AMSPQLHNI (SEQ ID NO: 4), GLAAPAPKL (SEQ ID NO: 5), GLPPHPAHL (SEQ ID NO: 6), GMPWPIPAV (SEQ ID NO: 7), SLQETSYAL (SEQ ID NO: 8), SLYPIACSL (SEQ ID NO: 9), SVLGHDFSV (SEQ ID NO: 10), VQDMIQTQV (SEQ ID NO: 11), ILDDWLRHL (SEQ ID NO: 12) and SLPSQHWSL (SEQ ID NO: 13).

The present invention relates to a peptide comprising or consisting of an epitope having a sequence selected in the group comprising or consisting of: LLLEATANL (SEQ ID NO: 1), SLTDLYLRI (SEQ ID NO: 2), AMSPQLHNI (SEQ ID NO: 4), GLAAPAPKL (SEQ ID NO: 5), GLPPHPAHL (SEQ ID NO: 6), GMPWPIPAV (SEQ ID NO: 7), SLQETSYAL (SEQ ID NO: 8), SLYPIACSL (SEQ ID NO: 9), SVLGHDFSV (SEQ ID NO: 10), VQDMIQTQV (SEQ ID NO: 11), ILDDWLRHL (SEQ ID NO: 12) and SLPSQHWSL (SEQ ID NO: 13).

In one embodiment, the peptide comprises or consists of an epitope having a sequence selected in the group comprising or consisting of LLLEATANL (SEQ ID NO: 1), SLTDLYLRI (SEQ ID NO: 2) and IMTASNWTL (SEQ ID NO: 3).

In one embodiment, the peptide comprises or consists of an epitope having a sequence selected in the group comprising or consisting of LLLEATANL (SEQ ID NO: 1) and SLTDLYLRI (SEQ ID NO: 2).

In one embodiment, the peptide comprises or consists of an epitope of sequence LLLEATANL (SEQ ID NO: 1).

In one embodiment, the peptide comprises or consists of an epitope of sequence SLTDLYLRI (SEQ ID NO: 2).

In one embodiment, the peptide comprises or consists of an epitope of sequence IMTASNWTL (SEQ ID NO: 3).

The present invention also relates to an expression vector inducing expression of one or more peptide(s) as described hereinabove.

Said vector may be especially a RNA vector, a DNA vector or plasmid, a viral vector or a bacterial vector. There can be integration of an expression cassette into the host cell genome or there can be no integration, depending on the nature of the vector and as this is well known to the skilled person. The expression vector or the expression cassette may further comprise elements necessary for the in vivo expression of the nucleic acid (polynucleotide) in a subject. For example, this may consist of an initiation codon (ATG), a stop codon and a promoter, as well as a polyadenylation sequence for certain vectors such as the plasmids and viral vectors other than poxviruses. The ATG may be placed at 5′ of the reading frame and a stop codon may be placed at 3′. As it is well-known, other elements making it possible to control the expression could be present, such as enhancer sequences, stabilizing sequences and signal sequences permitting the secretion of the peptide.

Regarding RNA vectors, said vectors may use, for example, non-replicating mRNA or virally derived, self-amplifying RNA. Conventional mRNA-based vectors may encode the peptide of interest and may contain 5′ and 3′ untranslated regions (UTRs). Self-amplifying RNAs may encode not only the peptide of interest but also the viral replication machinery that enables intracellular RNA amplification and abundant protein expression.

Examples of viral vectors, include, without limitation, lentivirus and retrovirus.

The present invention also relates to a cytotoxic T lymphocyte (CTL) of a subject treated with one or more peptide(s) as described hereinabove.

The present invention also relates to a cytotoxic T lymphocyte (CTL) of a subject treated with one or more expression vector(s) as described hereinabove.

The present invention also relates to a cytotoxic T lymphocyte (CTL) generated in vitro by stimulation of T cells with one or more peptide(s) or one or more expression vector(s) as described hereinabove.

The present invention also relates to a T-cell receptor (TCR) recognizing a peptide as described hereinabove.

The present invention also relates to an engineered T cell expressing a TCR (i.e., a TCR-engineered T cells) recognizing a peptide as described hereinabove.

The process of preparing these T cells is known from the skilled person. It may be the following: (i) TCR a and B chains are isolated from T cells recognizing peptides as described hereinabove and inserted into a vector; (ii) T cells isolated from the peripheral blood of a patient or a donor are modified with such a vector to encode the desired TCRαβ sequences; (iii) these modified T cells are then expanded in vitro to obtain sufficient numbers for treatment and administered into the patient. Of note, TCR sequences can be modified for optimization of TCR affinity.

The present invention also relates to one or more peptide(s), one or more expression vector(s), one or more CTL(s), or one or more engineered T cell(s) as described hereinabove for use as a vaccine.

The present invention also relates to one or more peptide(s), one or more expression vector(s), one or more CTL(s), or one or more engineered T cell(s) as described hereinabove for use as a medicament.

The present invention also relates to one or more peptide(s), one or more expression vector(s), one or more CTL(s), or one or more engineered T cell(s) as described hereinabove for use in treating or preventing at least one cancer in a subject in need thereof.

In one embodiment, the one or more peptide(s), one or more expression vector(s), one or more CTL(s), or one or more engineered T cell(s) as described hereinabove is for use in treating or preventing at least one, at least two, or more cancers.

In one embodiment, the at least one cancer is a c-myc associated cancer. In one embodiment, the at least one cancer is a IGF1R associated cancer.

Examples of c-myc and IGF1R associated cancers are provided hereinabove.

In one embodiment, the at least one cancer is selected from the group comprising or consisting of breast cancer, including triple negative breast cancer, ovarian cancer, melanoma, sarcoma, teratocarcinoma, colon cancer, prostate cancer, bladder cancer, lung cancer, including non-small cell lung carcinoma and small cell lung carcinoma, head and neck cancer, colorectal cancer, glioblastoma, leukemias, lymphomas and other solid tumors and hematological malignancies.

In one embodiment, the at least one cancer is colon cancer. In one embodiment, the at least one cancer is breast cancer.

In one embodiment, the one or more peptide(s), one or more expression vector(s), one or more CTL(s), or one or more engineered T cell(s) as described hereinabove is for use in treating or preventing at least one cancer, wherein the cancer is breast or colon cancer.

In one embodiment, the one or more peptide(s), one or more expression vector(s), one or more CTL(s), or one or more engineered T cell(s) as described hereinabove is for use in treating or preventing breast and colon cancers.

The present invention also relates to a method for treating or preventing at least one cancer in a subject in need thereof, wherein said method comprises the administration of one or more peptide(s), one or more expression vector(s), one or more CTL(s), or one or more engineered T cell(s) as described hereinabove in said subject.

In one embodiment, the method of the present invention is for treating or preventing at least one, at least two or more cancers as defined herein.

In one embodiment, the method of the present invention is for treating or preventing breast and/or colon cancer(s).

The present invention also relates to one or more peptide(s), one or more expression vector(s), one or more CTL(s), or one or more engineered T cell(s) as described hereinabove for the manufacture of a medicament for treating or preventing at least one cancer in in a subject in need thereof.

In one embodiment, the medicament is for treating or preventing at least one, at least two or more cancers as defined herein.

In one embodiment, the medicament is for treating or preventing breast and/or colon cancer(s).

In one embodiment, the one or more peptide(s), one or more expression vector(s), as described hereinabove induce an immune response, such as a T cell response.

One of ordinary skill would know various assays to determine whether an immune response against a tumor-associated epitope was generated. Various B lymphocyte and T lymphocyte assays are well known, such as ELISAs, cytotoxic T lymphocyte (CTL) assays, such as chromium release assays, cytometry-based assays or real-time cytotoxicity assays, proliferation assays using peripheral blood lymphocytes (PBL), tetramer assays, and cytokine production assays.

Thus, the present invention also relates to a method for inducing an immune response in a subject in need thereof, wherein said method comprises the administration of one or more peptide(s), or one or more expression vector(s), as described hereinabove in said subject.

In one embodiment, the one or more peptide(s), one or more expression vector(s), one or more CTL(s), or one or more engineered T cell(s) as described hereinabove reduces the number of tumor cells in vivo. Thus, the present invention also relates to an in vivo method for reducing the number of tumor cells, comprising administering to a subject in need thereof, a therapeutically effective amount of one or more peptide(s), one or more expression vector(s), one or more CTL(s), or one or more engineered T cell(s) as described hereinabove.

In one embodiment, the one or more peptide(s), one or more expression vector(s), one or more CTL(s), or one or more engineered T cell(s) as described hereinabove reduces the tumor volume in vivo. Thus, the present invention also relates to a method for reducing the tumor volume in vivo, comprising administering to a subject in need thereof, a therapeutically effective amount of one or more peptide(s), one or more expression vector(s), one or more CTL(s), or one or more engineered T cell(s) as described hereinabove.

In one embodiment, the one or more peptide(s), one or more expression vector(s), one or more CTL(s), or one or more engineered T cell(s) as described hereinabove has cytotoxic activity against tumor cells but no cytotoxic effect against normal cells (i.e. non-tumor cells).

In one embodiment, the one or more peptide(s), one or more expression vector(s), one or more CTL(s), or one or more engineered T cell(s) as described hereinabove is/are administered at a therapeutically effective amount.

It will be however understood that the total daily usage of one or more peptide(s), one or more expression vector(s), one or more CTL(s), or one or more engineered T cell(s) as described hereinabove will be decided by the attending physician within the scope of sound medical judgment.

The specific dose for any particular subject will depend upon a variety of factors including the symptom being treated and the severity of the symptom; activity of the specific compound employed; the specific composition employed, the age, body weight, general health, sex and diet of the patient; the time of administration, route of administration, and rate of excretion of the specific compound employed; the duration of the treatment; drugs used in combination or coincidental with the specific compounds employed; and like factors well known in the medical arts.

For use in administration to a subject, the one or more peptide(s), one or more expression vector(s), one or more CTL(s) or one or more engineered T cell(s) is/are to be formulated for administration to the subject.

The one or more peptide(s), or one or more expression vector(s), as described hereinabove may be administered by enteral or parenteral route of administration.

The one or more CTL(s) or one or more engineered T cell(s) as described hereinabove may be administered by a parenteral route of administration.

The enteral route may be selected from the group consisting of buccal route (including perlingual route and sublingual route), oral route and rectal route.

The parenteral includes any route that is not enteral. The parenteral route may be selected from the group consisting of epicutaneous route, transdermal route, intradermal route, subcutaneous route, nasal route, intramuscular route, intraocular route, intravitreal route, and intravitreal route.

SEQUENCE LISTING

| SEQ ID | |
|---|---|
| NO: | Sequence |
| 1 | LLLEATANL |
| 2 | SLTDLYLRI |
| 3 | IMTASNWTL |
| 4 | AMSPQLHNI |
| 5 | GLAAPAPKL |
| 6 | GLPPHPAHL |
| 7 | GMPWPIPAV |
| 8 | SLQETSYAL |
| 9 | SLYPIACSL |
| 10 | SVLGHDFSV |
| 11 | VQDMIQTQV |
| 12 | ILDDWLRHL |
| 13 | SLPSQHWSL |
| 14 | FLLMPLSFL |
| 15 | IILGIVFLL |
| 16 | SLDHLLLEA |
| 17 | FLWKRGRLL |
| 18 | ALLDGVLPA |
| 19 | LLFKVDFFL |
| 20 | RLGAAVFLL |
| 21 | RLLAKGQSL |
| 22 | SQPPPSLFV |
| 23 | ALLRCGHTL |
| 24 | GQASVPLFL |
| 25 | KLQTLLASI |
| 26 | LLASILFYI |
| 27 | LLVSTGVTV |
| 28 | QMQPHNLGV |
| 29 | SLFKLQTLL |
| 30 | VMFFKSQHL |
| 31 | ALADEWRNL |
| 32 | ALMISLGSV |
| 33 | ALWQDHTEI |
| 34 | AQWPAPRLV |
| 35 | CLLSKPVRL |
| 36 | FLFSICKQL |
| 37 | FLLTPRNFL |
| 38 | FMMITAYTV |
| 39 | FSIELLFSV |
| 40 | GLFFSLMFL |
| 41 | GLKPWTQYA |
| 42 | KLFGFCFQL |
| 43 | KLISELRRI |
| 44 | KLSELLMSF |
| 45 | LLFSVNREV |
| 46 | LLLAGGPGL |
| 47 | LLPGGLLLL |
| 48 | LLPPAPLVV |
| 49 | LLQALMISL |
| 50 | LLVISLWSV |
| 51 | LLWKLISEL |
| 52 | QIIQLVIRV |
| 53 | RLAPLFQQL |
| 54 | SLKDGVFTT |
| 55 | SLSWETPGV |
| 56 | SLWPHPTTV |
| 57 | SMMGRMPAA |
| 58 | SVHPTAPAV |
| 59 | SVPKHVWEA |
| 60 | VLFKLSELL |
| 61 | VLFSILVST |
| 62 | ALLTFSLFL |
| 63 | ALPGLVQRA |
| 64 | FIFGLHLRL |
| 65 | FITEKLPQV |
| 66 | FLFSRWILL |
| 67 | FLVKKKFFV |
| 68 | GLCSLPPLL |
| 69 | GLLRGMSRL |
| 70 | LLHSLSTKV |
| 71 | LLLERDPSL |
| 72 | LLLGKCLGV |
| 73 | RVTDVILFL |
| 74 | SLAPDSRPV |
| 75 | SLISVENRA |
| 76 | SLSHSVFPL |
| 77 | SMLDHETFA |
| 78 | SVDEKNFKM |
| 79 | TLLSIPNYV |
| 80 | TLSFFTLKL |
| 81 | VQMEPTHFV |
| 82 | YLSPFGHEI |
| 83 | MRRHRPPPA |
| | ATLRRNKKM |
| | RKKSMLFLW |
| | KRGRLLAKG |
| | QSLDHLLLE |
| | ATANLLTAH |
| | WSSRGATSP |
| | HISTTTQRL |
| | PPLGRTILL |
| | PRGSSWTVS |
| | ES |
| 84 | RLPPLGRTI |
| 85 | LPRGSSWTV |
| 86 | LPPLGRTIL |
| 87 | ATANLLTAH |
| 88 | TQRLPPLGR |
| 89 | ILLPRGSSW |
| 90 | LLEATANLL |
| 91 | KSMLFLWKR |
| 92 | MLFLWKRGR |
| 93 | KMRKKSMLF |
| 94 | HRPPPAATL |
| 95 | PPLGRTILL |
| 96 | SPHISTTTQ |
| 97 | GQSLDHLLL |
| 98 | RPPPAATLR |

### BRIEF DESCRIPTION OF THE DRAWINGS

FIGS. 1A-C are a combination of a schema and tables. FIG. 1A: Schematic representation of the bioinformatic prediction pipeline to identify potential neoepitopes derived from non-canonical translation of major oncogenes. All ≥9-mer peptides that may result from non-canonical translation are predicted (1). Potential HLA-A*02:01 strong binder 9-mer epitopes from those predicted peptides are identified (2). Epitopes with perfect sequence homology to human proteome are removed from selection (3). Proteomic mass spectrometry databases from tumor and normal tissues are used to filter predicted HLA-A*02:01 strong binder epitopes (4). FIG. 1B: List of all predicted HLA-A*02-01 strong binder epitopes, for MYC (left column) and IGF1R (right column) analysis, using netMHCpan. FIG. 1C: List of predicted HLA-A*02-01 strong binder epitopes found at least 1 time “confident” in tumor tissues proteomic mass spectrometry databases but “not confident” in normal tissues database, for MYC (left column) and IGF1R (right column) analysis, using Pepquery. FIG. 1D: List of predicted class I HLA strong binder epitopes for the MYC non-canonical peptide containing PR3 epitope, using netMHCpan and MHCflurry. FIG. 1E: List of predicted class I HLA weak binder epitopes for the MYC non-canonical peptide containing PR3 epitope, using netMHCpan and MHCflurry. FIG. 1F: Distribution of the binding score for the predicted class I HLA strong (up panel) and weak (down panel) binder epitopes for the MYC non-canonical peptide containing PR3 epitope.

FIGS. 2A-B are a combination of graphs. FIG. 2A: Representative plots of dextramer staining of CD8+ T cells after 12 days specific peptide pulsed (upper row) or unpulsed (lower row) MoDCs: CD8+ naive T cells priming. FIG. 2B: Schematic representation of dextramer analysis after 12 days MoDCs: CD8+ naive T cells priming for 12 healthy donors.

FIG. 3 shows representative plots of dextramer staining of CD8+ T cells after 12 days MoDCs: CD8+ naive T cells priming (left panel) and after sorting and expansion of peptide specific CD8+ T cells and unspecific counterpart (right panel).

FIGS. 4A-B are a combination of two histograms. FIG. 4A: Representative graphic of the percentage of 4-1BB expression of peptide specific or unspecific CD8+ T cells after contact with T2 cells pulsed with irrelevant (non-specific) or specific peptide, on 3 donors for PR3 condition and one donor for PR5 condition. FIG. 4B: Representative graphic of the percentage of CFSE+ T2 cell death pulsed with irrelevant (non-specific) or specific peptide after contact with peptide specific or unspecific CD8+ T cells, on 2 donors for PR3 condition and one donor for PR5 condition.

FIGS. 5A-B are a combination of two histograms. Representative graphic of IFN gamma (A.) or TNF alpha (B.) concentrations produced by peptide specific or unspecific CD8+ T cells after contact with T2 cells pulsed with irrelevant (non-specific) or specific peptide.

FIGS. 6A-B are a combination of two graphs representing real-time cell death quantification by Incucyte of MDA-MB-231 (A.) or HCT116 (B.) cell lines (pulsed or not with PR3 peptide) co-cultured with PR3-specific CD8+ T cells or their negative counterpart (dextramer-neg T cells).

FIG. 7A-C are a combination of graphs showing transition of PR3 (LLLEATANL) epitope in tumor cell lines (A. MDA-MB-231 and B. OVCAR-3) or a panel of HLA-A2+ normal human primary cells (C.), using the Valid-NEO method builder bioinformatic pipeline (Complete Omics Inc., MD, USA).

FIG. 8 is a histogram showing IFN gamma concentrations produced by PR3-specific CD8+ T cells or their negative counterpart (dextramer-neg T cells) after 48 h co-culture with either HLA-A2+ normal human primary cells (cardiomyocytes, bronchial epithelial cells or keratinocytes) or tumor cell lines (MDA-MB-231).

### EXAMPLES

The present invention is further illustrated by the following examples.

Bioinformatic Prediction Pipeline to Identify Potential Neoepitopes Derived from Non-Canonical Translation of Major Oncogenes

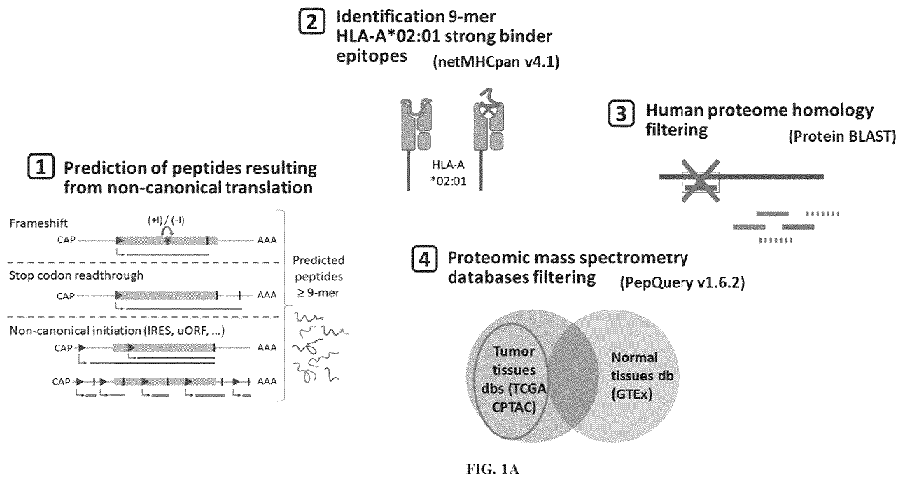

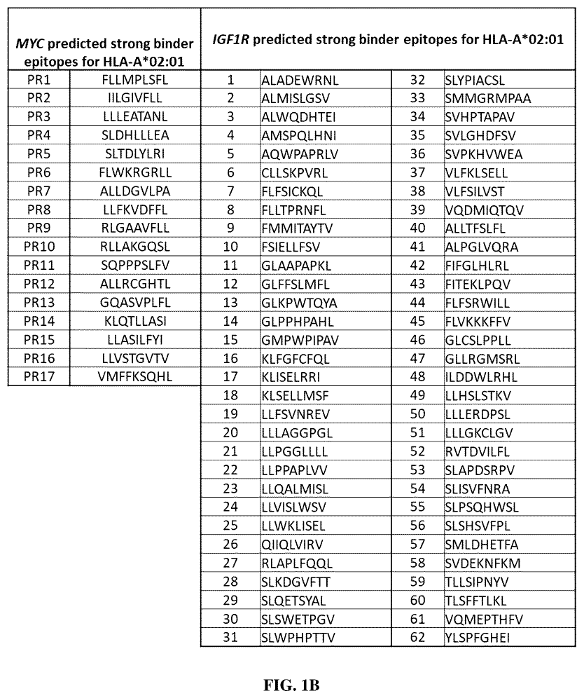

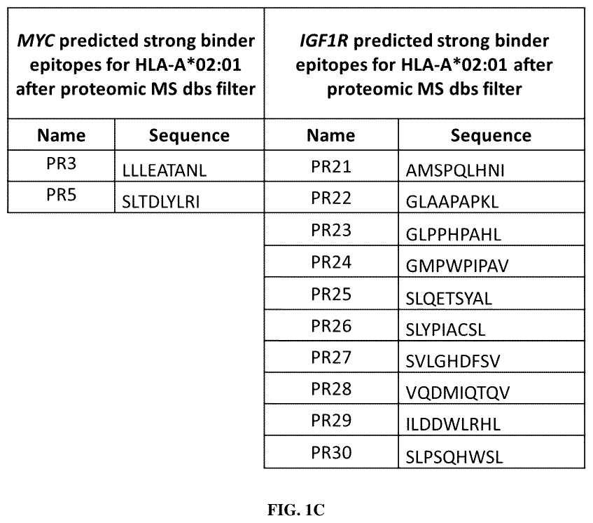

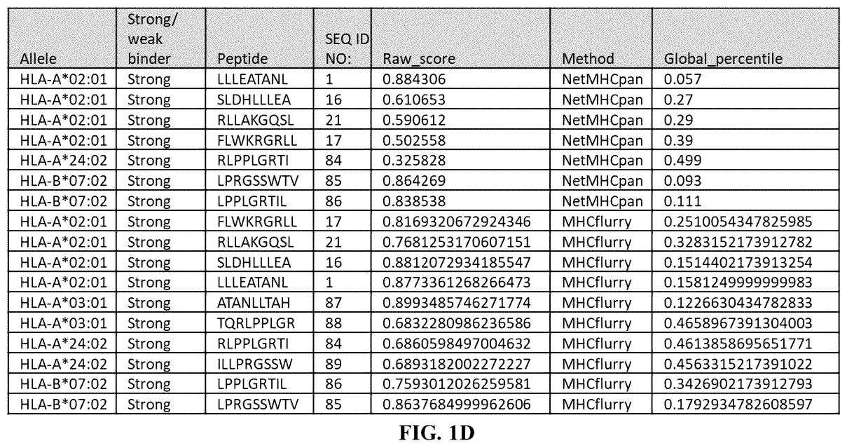

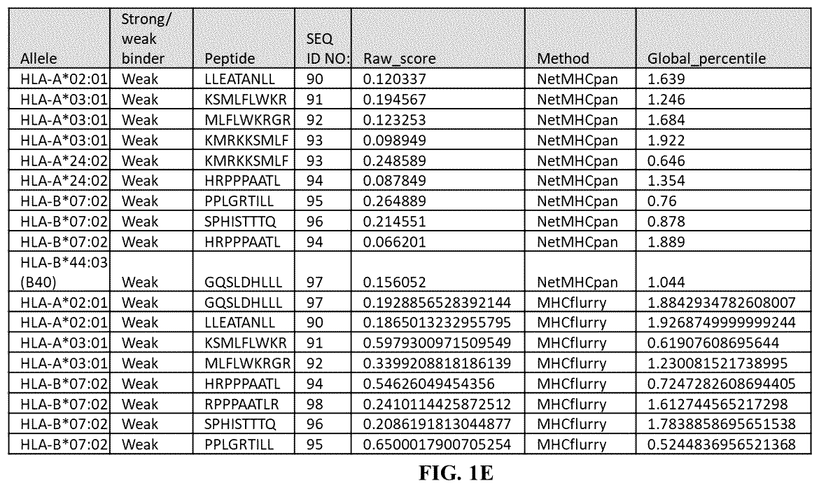

All transcripts of two major oncogenes, MYC and IGF1R, have been identified using the genome browser Ensembl. For each transcript of each oncogene, a bioinformatic pipeline have been set up to predict all peptides sequences with size superior or equal to 9-mer that could result from a defect in translation (frameshift (+1) or (−1), defect in termination of translation, such as stop codon readthrough) and/or any non-canonical initiation (uORF, IRES, translation in 5′UTR or 3′UTR). Among those sequences, 9-mer strong binder epitopes for HLA-A*02:01 (% rank≤0.5) have been predicted using epitope prediction tool (netMHCpan v4.1). Any potential epitope with a perfect sequence homology to the human proteome was removed from the previous selection (BLAST protein, refseq_protein database) (FIG. 1A). With this analysis, 17 potential strong binder epitopes for HLA-A*02:01 have been identified for MYC oncogene analysis, and 62 for IGF1R oncogene analysis (FIG. 1B).

### Proteomic Mass Spectrometry (MS) Databases Filtering

A targeted peptide search engine has been used to filter potential translated epitopes based on mass spectrometry-based proteomics datasets (PepQuery V1.6.2). Epitopes found at least 1 time confident in proteomic mass spectrometry databases for breast or colon cancers (patient tumor datasets: TCGA and CPTAC) but not confident in normal tissue proteomic database (GTEx) were selected (FIG. 1A). For MYC oncogene analysis, 2 epitopes (PR3 and PR5) among the 17 previously predicted were selected. For IGF1R oncogene analysis, 10 epitopes among the 62 previously predicted were selected (FIG. 1C).

Prediction of Class I HLA Epitopes Derived from One of the MYC Non-Canonical Predicted Peptide, Containing PR3 Epitope

Using the previous bioinformatic prediction pipeline, we identify several amino acids sequences derived from the non-canonical translation on MYC oncogene. One of them (sequence with SEQ ID NO: 83) was predicted to contain the PR3 sequence and to lead to the epitope PR3 after processing.

SEQ ID NO: 83
MRRHRPPPAATLRRNKKMRKKSMLFLWKRGRLLAKGQSLDHLLLEA
TANLLTAHWSSRGATSPHISTTTQRLPPLGRTILLPRGSSWTVSES

Using this sequence, we predicted all 9-mer strong and weak binder epitopes for frequent class I HLA class I alleles using epitope prediction tool (netMHCpan v4.1 or MHCFlurry) (FIGS. 1D-1F).

### Peptide Synthesis

Peptides were synthetized (JPT peptide technology, Germany) and their identities were confirmed by mass spectrometry by the seller. Purity>95% was expected and determined by high-performance liquid chromatography. Lyophilized peptides were dissolved in deionized water<5% DMSO, aliquoted and conserved at −20° C. until use.

### PBMCs Priming Assay on HLA-A*02:01 Healthy Donors

PBMCs were obtained by Ficoll density gradient centrifugation of blood from HLA-A*02:01 healthy donors (“Etablissement Français du Sang”, EFS, Lyon). For priming assay, PBMCs were rapidly thawed at 37° C., extensively washed and let at room temperature for 2 h before assessing their viability. Monocytes were isolated by positive selection of CD14+ cells (Miltenyi). Monocytes-derived dendritic cells (MoDCs) were generated from 4-days culture of CD14+ monocytes in complete RPMI (RPMI medium with 10% fetal Calf Serum (FCS) and 1% Penicillin Streptomycin (PS)) supplemented with recombinant human IL-4 (10 ng/ml) and recombinant human GM-CSF (800 UI/ml at day 1; 1600 UI/ml at day 3). MoDCs were then maturated and pulsed (or not for negative control) overnight (18 h) with peptides (10 μg/ml for specific peptides, 2.5 μg/ml for positive control), IL-4 (10 ng/ml), GM-CSF (800 UI/ml), TNFα (20 ng/ml) and Poly-IC (40 μg/ml). In the meantime, CD8+ naive T cells were isolated from thawed autologous PBMCs (Miltenyi kit) and cultured overnight in AIM-V medium with 5% human serum AB (sAB) and 1% PS, supplemented with recombinant human IL-7 (5 ng/ml). Next day, pulsed MoDC were co-cultured with CD8+ naive T cells for 12 days (MoDC: CD8+ naive T cells ratio 1:4) in 48-wells plate with AIM-V+5% sAB+1% PS supplemented with human recombinant IL-21 (60 ng/ml). Over the 12 days, cells were amplified from 48-wells plate to 12-wells and then 6-wells plate, and AIM-V 5% sAB+1% PS supplemented with IL-7 and IL-15 (10 ng/ml each at day 3 and 5; 20 ng/ml at day 7 and 10) was added. Optimized peptide MARTI (ELAGIGILTV) was used as positive control for priming assay.

### Dextramer Analysis

On day 12, peptide specific CD8+ T cells were identified using dextramer staining (Immudex). For each peptide condition, peptide pulsed cells and unpulsed cells were stained with corresponding dextramer. For the staining, 3·10⁶ cells were put in polypropylene tubes and washed with FACS buffer. Cells were stained with 8 μL of dextramer for 10 min at room temperature in the dark, then with 1/400 diluted Zombi Near Infra-Red (NIR) for 10 more min in the dark to assess viability (Biolegend). Anti-CD3 BV421 and anti-CD8 FITC (Biolegend) antibodies were added for 20 min in the dark at 4° C. Cells were then washed twice with FACS buffer and resuspend in FACS buffer for Flow Cytometry analysis (FACS Fortessa BD).

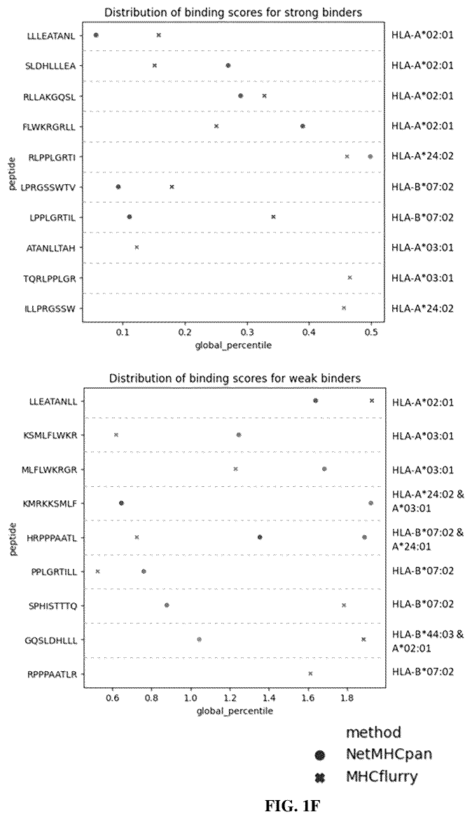

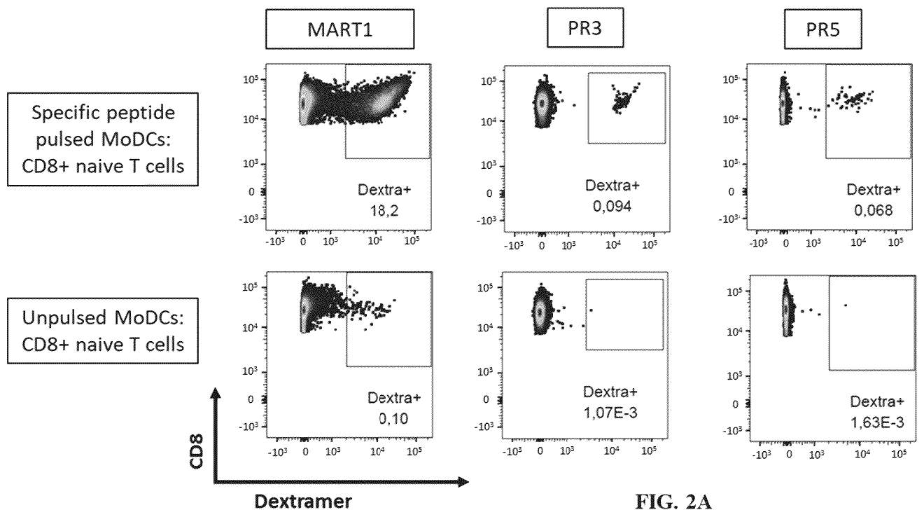

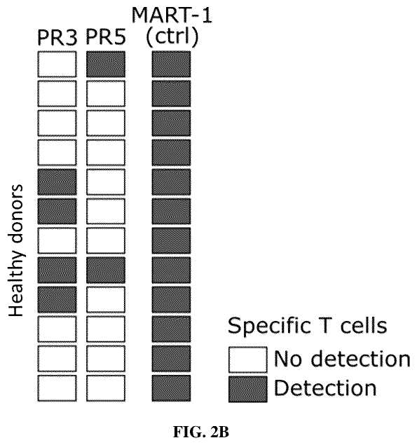

Results in FIG. 2A show CD8+ T cells stained by dextramer in unpulsed (lower row) versus peptide specific-pulsed (upper row) primed cells for each peptide condition. For MARTI positive control, up to 18.2% of CD8+ T cells resulted positive after stimulation with MARTI peptide vs. 0.1% in unpulsed condition. Interestingly, condition stimulated with specific epitopes (PR3 and PR5) resulted in a 0.094% and 0.068% of dextramer positive CD8+ T cells for PR3 and PR5 respectively vs. 0.001% and 0.002% in unpulsed conditions. Results obtained on 12 different donors are summarized in FIG. 2B, with dark box when peptide specific CD8+ T cells were detected after priming and white box otherwise. Among them, priming of peptide specific CD8+ T cells was identified in 4 donors for PR3 and 2 donors for PR5.

### Peptide Specific CD8+ T Cells Sorting

After 12 days MoDCs: CD8+ naive T cells priming and validation of peptide specific CD8+ T cells by dextramer staining analysis, cells were sorted using two different protocols.

For the first one, cells from MoDCs: CD8+ naive T cells priming were stained using the same protocol as dextramer analysis, without anti-CD3 antibody. Dextramer positive cells corresponding to peptide-specific CD8+ T cells and negative cells corresponding to unspecific counterpart were sorted using BD FACSAria™ Cell Sorter, after gating on lived CD8+ T cells.

For the second protocol, peptide-specific CD8+ T cells were sorted using peptide-specific monomers coupled with magnetics beads. In 1.5 ml tube, 10 μL of peptide specific biotinylated monomer at 100 μg/ml (P2R Facility, Nantes, France) are incubated on rotary shaker at room temperature for 1 h with 10 μL of dynabeads M-280 streptavidin in PBS 1×0.1% BSA (100 μL final volume). Magnetic peptide-specific monomers formed are then washed 3 times with PBS 1×0.1% BS using DynaMag Spin Magnet. 5·10⁶ cells from MoDC: CD8+ naive T cells priming are washed and resuspended in 500 μL of PBS 1×0.1% BSA. They are mixed to previously formed magnetic monomers and incubated for 4 h on rotary shaker at room temperature. Cells are then washed 8 to 10 times with PBS 1×0.1% BSA using DynaMag Spin Magnet. The only sorted fraction in this protocol is the peptide-specific CD8+ T cells attached to magnetic peptide specific monomers.

### Feeding Protocol

Peptide-specific CD8+ sorted T cells and unspecific counterpart were expanded on a feeder composed by 35 Gy-irradiated allogenic PBMCs and B-lymphoblastic cell lines in a ratio 10:1. Feeder cells were plated in a 96-well round bottom plate at a concentration of 0.10×10⁶ cells per well in RPMI 8% sAB 1% PS, supplemented with PHA-L (1.5 μg/mL), human recombinant IL-2 (150 IU/mL) and human recombinant IL-7 (10 ng/ml). Up to 5×103 sorted cells were added per well. Cells were cultured for 14 days. From day 5 and every two days (day 5, 7, 9 and 12), half medium was replaced or each well was split in half depending on proliferation with RPMI 8% sAB 1% PS, supplemented with human recombinant IL-2 (300 UI/ml), human recombinant IL-7 (20 ng/ml) and human recombinant IL-15 (20 ng/ml). After 12 days, the purity of the specific versus unspecific fraction was evaluated. Cells were used for cytotoxicity experiments if there were ≥65% of dextramer positive CD8+ T cells in the positive fraction and <0.5% in the negative fraction.

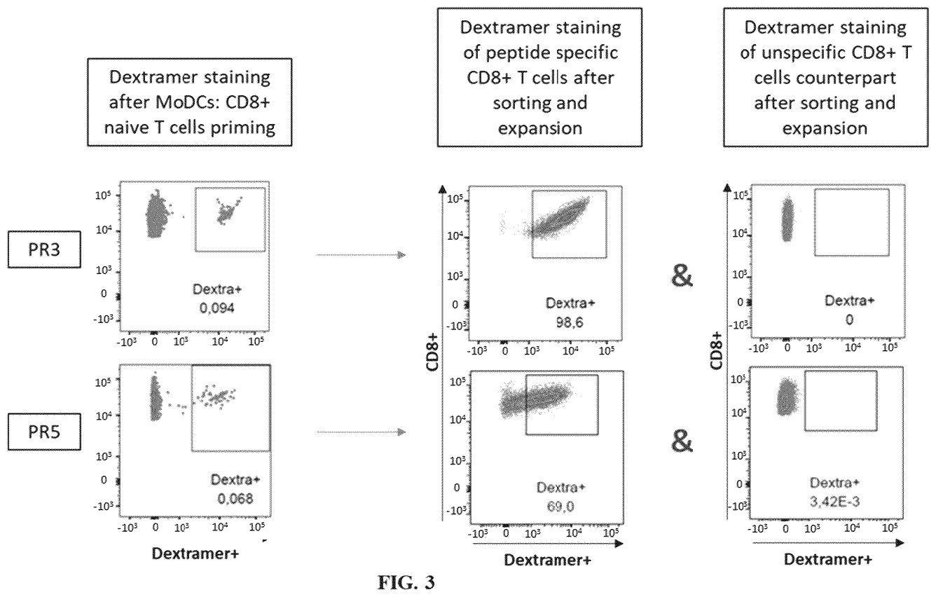

Results in FIG. 3 show dextramer staining of CD8+ T cells after 12 days MoDCs: CD8+ naive T cells priming and after sorting and expansion of the peptide-specific CD8+ T cells or unspecific counterpart. CD8+ T cell populations specific at 98.6% for PR3 peptide and 69% for PR5 peptide were sorted and amplified from cells resulting from peptide pulsed MoDCs: CD8+ T cells priming.

Cytotoxicity and Functional Assay with T2 Cells

T2 (SD cell line) are a lymphoblast cell line deficient in the transporter associated antigen processing (TAP) protein, and therefore cannot present endogenous peptides on the class I MHC but can be used to monitor the Cytotoxic T Lymphocyte (CTL) response to an exogenous antigen of interest in a non-competitive environment.

T2 cells are first stained with CFSE cell division tracker kit (Biolegend) for 13 min at 37° C. and then washed 3 times. CFSE stained T2 cells are pulsed with irrelevant (non-specific) or specific peptide for 2 hours at 37° C. After extensive washed, CFSE pulsed T2 cells are resuspended in the corresponding medium of T cells, RPMI+8% sAB+1% PS. A resting is performed for T cells. First, CD8+ T cells are resuspended in RPMI 8% sAB+1% PS supplemented with 50 UI/ml of human recombinant IL-2 overnight at 37° C. The next day, CD8+ T cells are resuspended in their corresponding medium at 37° C. for 2 h without cytokine supplementation. Then, CFSE pulsed T2 cells and T cells are co-cultured (T2: CD8+ T cells ratio 1:10) in 96-well U-bottom plate in duplicate. After 24 h, supernatent is collected for further ELISA analysis and cells are pooled in V-well plate and washed with FACS buffer. For each condition, a mix of antibody is added containing Zombi NIR (dil 1/400), anti-CD3 BV421, anti-CD8 APC and anti-human CD137 (4-1BB) Pe-Dazzle594 (Biolegend) antibodies for 30 min at 4° C. in the dark. Cells are washed and resuspended in FACS buffer before Flow Cytometry analysis (FACS Fortessa BD).

FIG. 4A shows the percentage of 4-1BB expression of peptide-specific or unspecific CD8+ T cells after contact with irrelevant or specific peptide pulsed T2 cells. FIG. 4B shows the percentage T2 cell death after contact with peptide-specific CD8+ T cells or unspecific counterpart. These results show a specific activation (A.) and a specific killing (B.) of PR3 and PR5 specific CD8+ T cells upon specific antigenic stimulation.

For ELISA analysis, IFN gamma Human Uncoated ELISA Kit (Invivogen) and TNF alpha Human Uncoated ELISA Kit (Invivogen) are used according to the manufacturer's instructions. Different dilutions of supernatent have been tested (dil 1/2, 1/5, 1/10 or 1/20) and the one which absorbance results were included in standard range were analyzed.

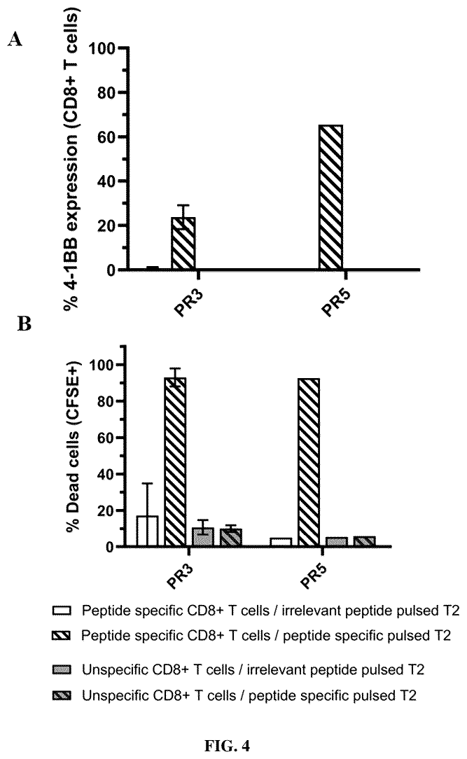

Results on FIG. 5 show cytokines (A. IFN gamma and B.TNF alpha) secretion quantification of peptide specific and unspecific CD8+ T cells after contact with irrelevant or specific peptide pulsed T2 cells. As previous results, there is a specific IFN gamma and TNF alpha secretion of PR3 and PR5 specific CD8+ T cells upon specific antigenic stimulation.

### Validation of Epitope Presentation by Mass Spectrometry

Epitope validation by mass spectrometry were performed by Complete Omics Inc. (MD, USA). In brief, a total of 20 million cells were lysed, and peptide-HLA complexes were immunoprecipitated using self-packed Valid-NEO neoantigen enrichment column preloaded with anti-human HLA-A, B, and C antibody clone W6/32 (BioXCell). After elution, dissociation, filtration, and cleanup, peptides were lyophilized before further analysis. Transition parameters for each epitope peptide were examined and curated through Valid-NEO method builder bioinformatic pipeline to exclude ions with excessive noise due to coelution with impurities and to boost up the detectability through recursive optimizations of significant ions.

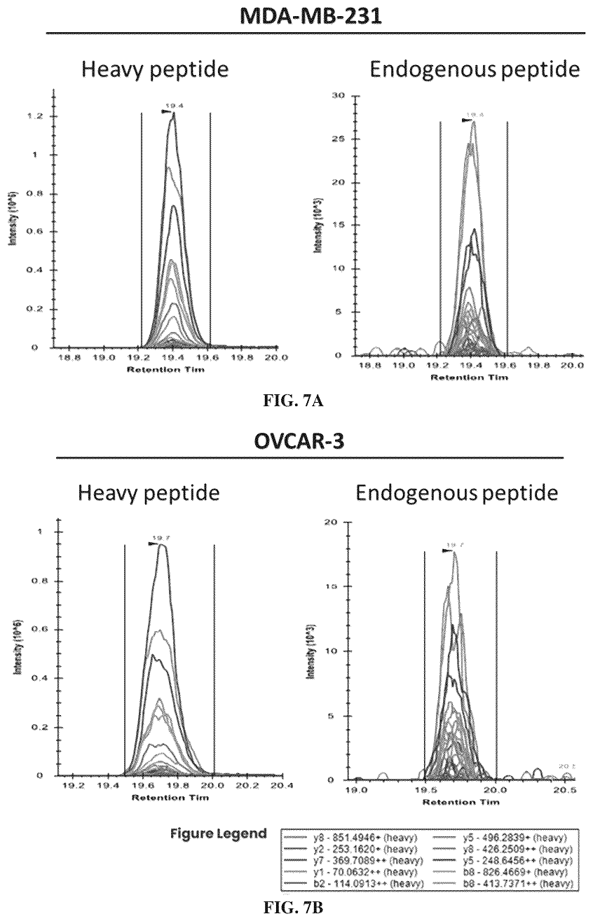

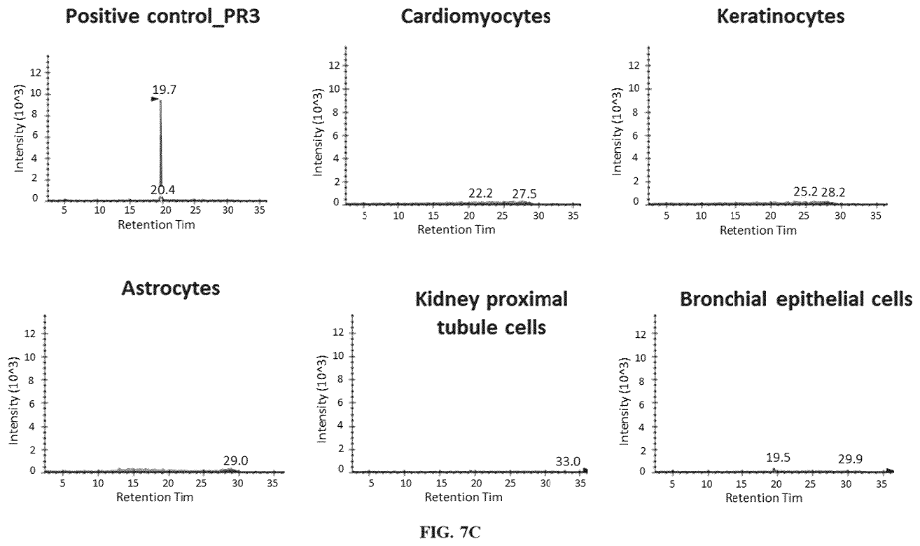

Results on FIGS. 7A-B show transition parameters for PR3 epitope in two tumor cell lines, MDA-MB-231 and OVCAR-3. Similar quantitative transitions were identified when the analysis was performed with the heavy peptide or peptides eluted from HLA of MDA-MB-231 or OVCAR-3 cell lines, confirming the presentation of PR3 epitope at the surface of both tumor cell lines.

In the contrary, no transitions were identified when analyzing a panel of HLA-A2+ normal human primary cells (cardiomyocytes, keratinocytes, astrocytes, kidney proximal tubule cells or bronchial epithelial cells, FIG. 7C), meaning that PR3 epitope was not presented by normal cells. These data suggest that PR3 epitope is a tumor specific and safe target.

### In Vitro Cytotoxicity of PR3 Specific T Cells Against Tumor Cell Lines Using InCucyte Technology

In order to monitor tumor cell death in real-time, we performed an immune cell killing assay using the IncuCyte technology. Tumor cell lines used as target were breast cancer cell line MDA-MB-231 and colon cancer cell line HCT116. For positive control condition, cell lines were pulsed with the specific peptide to artificially present target epitope on class I MHC. The day before the experiment, tumor cell lines are seeded at 5 000 cells/well in 96-well flat-bottom plate. For previous T cells, a resting is performed as for cytotoxicity and functional analysis on T2 cells, with a resting overnight with cytokines supplementation and a 2 h resting the next day without cytokines. In the meantime, tumor cell lines are pulsed or not with specific peptide at 10 μg/ml for 2 h and then washed 3 times. T cells are added in corresponding wells (CD8+ T cells: tumor cell lines ratio 2:1). For tumor cell death control condition, medium is added with DMSO (20% final). Finally, Incucyte® Cytotox Green for Counting Dead Cells (Sartorius) is added to reach a final concentration of 250 nM per well. This reagent enters the cells when the plasma membrane integrity diminished, yielding a 100-1000-fold increase in fluorescence upon binding to deoxyribonucleic acid (DNA). A 56-hour live imaging was performed at 37° C. 5% CO2 with Incucyte Zoom. For the analysis, the number of dead tumor cells per well is calculated by the evaluating the number of green fluorescence tumor cells per well.

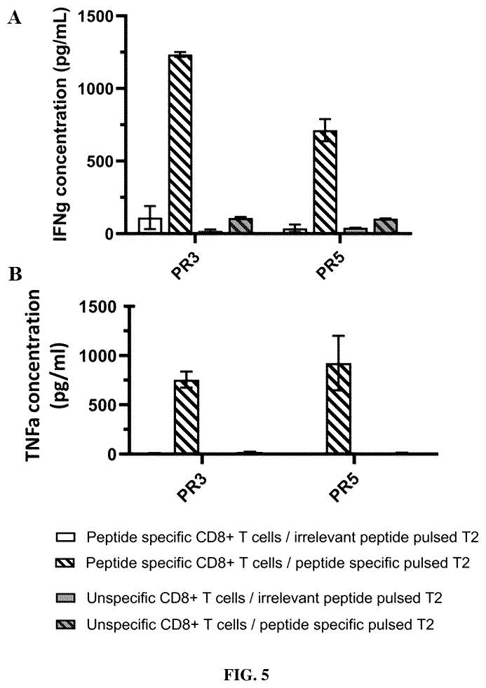

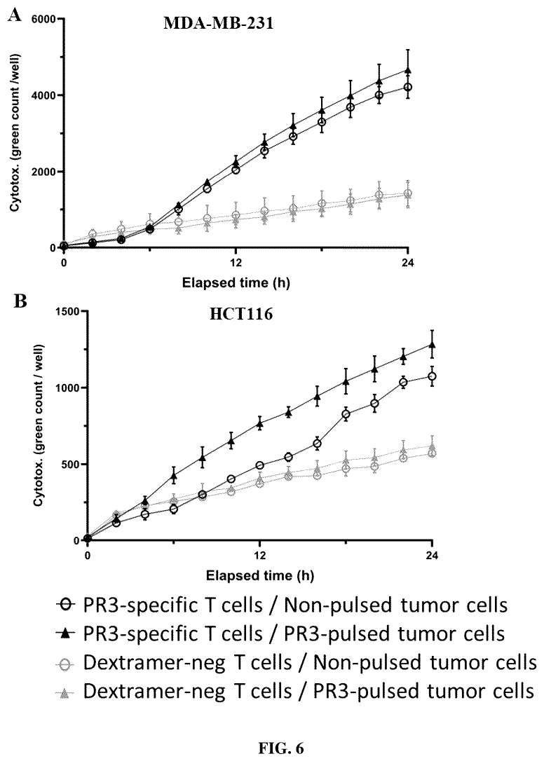

Results on FIG. 6 show kinetics of tumor cell death after co-culture with PR3-specific CD8+ T cells or with unspecific counterpart (dextramer-neg T cells), for MDA-MB-231 cell line (A.) or HCT116 cell line (B.), as well as a representative image of each condition at 24 h. For both cell line conditions, these results show an increase in cell death when tumor cells are co-cultured with PR3-specific CD8+ T cells in comparison to their negative counterpart. A further increase of cell death was observed when tumor cells were pulsed with PR3 and co-cultured with PR3-specific CD8+ T cells, in agreement with an epitope specific reactivity.

### Safety Analysis of PR3 Specific T Cells Against HLA-A2+ Normal Human Primary Cells

HLA-A2+ normal human primary cells were used to evaluate the safety of PR3 specific T cells, including cardiomyocytes, bronchial epithelial cells and keratinocytes (Promocell). Normal human primary cells and tumor cell line MDA-MB-231 as a positive control for cytotoxicity were plated at 5 000 cells per well in a 96-well flat-bottom plate. For T cells, a resting is performed as for cytotoxicity and functional analysis on T2 cells, with a resting overnight with cytokine supplementation and a 2 h resting the next day without cytokine. T cells are added in corresponding wells (CD8+ T cells: tumor cell lines ratio 10:1). After 48 h co-culture, supernatent are collected for further ELISA analysis using the IFN gamma Human Uncoated ELISA Kit (Invivogen) as described before.

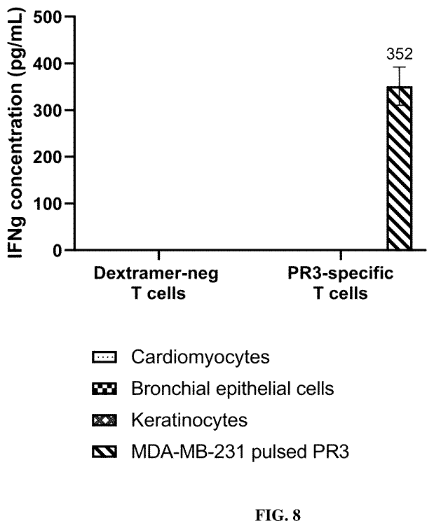

Results in FIG. 8 show IFN gamma secretion quantification of PR3-specific CD8+ T cells or with unspecific counterpart (dextramer-neg T cells) after 48 h co-culture with normal human primary cells and tumor cell line MDA-MB-231 as a positive control for cytotoxicity. No IFNg secretion is detected when PR3-specific CD8+ T cells are co-cultured with any of the normal human primary cells tested, suggesting the safety of the product. We observe a secretion of INFg when PR3-specific CD8+ T cells are co-cultured with tumor cell line MDA-MB-231 (positive control).

Altogether, these experiments show that PR3-specific CD8+ T cells specifically recognize and are functional against target cells presenting the cognate peptide (T2 cells) and specifically recognize and kill tumor cells expressing endogenously peptide derived from non-canonical translation (MDA-MB-231 and HCT116), without any toxicity against normal human primary cells.

1-15. (canceled)

16. A method for identifying one or several shared cancer epitope(s), wherein said method comprises the following step:
(a) identifying peptides derived from non-canonical initiation and/or termination of translation of a given gene and selecting among the identified peptides one or several shared cancer epitope(s).

17. The method according to claim 16, wherein said step (a) comprises the following steps:
(a₁) predicting the peptides derived from non-canonical initiation and/or termination of translation of a given gene,
(a₂) identifying among the predicted peptides identified in step (a₁) the 8- to 15-mer peptides (i.e. epitopes) that bind to MHC class I molecules,
(a₃) identifying among the sequences of the 8- to 15-mer epitopes identified in step (a₂) the epitopes that are found in healthy subjects and/or healthy tissues and excluding said epitopes, and
(a₄) selecting among the remaining epitopes of step (a₃) one or several epitope(s) found in at least one cancer.

18. The method according to claim 16, wherein the gene is an oncogene.

19. The method according to claim 18, wherein the oncogene is selected from the group comprising or consisting of c-myc, and IGF1R.

20. The method according to claim 18, wherein the oncogene is c-myc.

21. The method according to claim 17, wherein the MHC class I molecules are HLA-A molecules.

22. The method according to claim 17, wherein the at least one cancer is a c-myc associated cancer or an IGF1R associated cancer.

23. The method according to claim 17, wherein the at least one cancer is breast cancer or colon cancer.

24. The method according to claim 16, wherein said method further comprises an in vitro validation of the selected epitope(s) after step (a).

25. The method according to claim 24, wherein said in vitro validation comprises at least one of the three following steps:
evaluating the induction of CD8+ T cell responses by the selected epitope(s),
evaluating the functionality of the CD8+ T cells specific for the selected epitope(s), and/or
evaluating the cytotoxicity of the CD8+ T cells specific for the selected epitope(s) in tumor cells and non-tumoral cells,
optionally, wherein said in vitro validation further comprises a step (iv) of evaluating the expression of the selected epitope(s) in tumor cells.

26. The method according to claim 25, wherein said in vitro validation comprises the three steps (i), (ii) and (iii), optionally wherein said in vitro validation further comprises a step (iv) of evaluating the expression of the selected epitope(s) in tumor cells.

27. The method according to claim 25, wherein the step (iv) of evaluating the expression of the selected epitope(s) in tumor cells is assessed by ribosome profiling or mass spectrometry.

28. A method for producing one or several shared cancer epitope(s), wherein said method comprises a step (a) as described in claim 16, and a step (b) of generating the epitope(s) selected in step (a).

29. A peptide comprising or consisting of an epitope identified by the method according to claim 16.

30. A peptide comprising or consisting of an epitope having a sequence selected in the group comprising of consisting of LLLEATANL (SEQ ID NO: 1), SLTDLYLRI (SEQ ID NO: 2), AMSPQLHNI (SEQ ID NO: 4), GLAAPAPKL (SEQ ID NO: 5), GLPPHPAHL (SEQ ID NO: 6), GMPWPIPAV (SEQ ID NO: 7), SLQETSYAL (SEQ ID NO: 8), SLYPIACSL (SEQ ID NO: 9), SVLGHDFSV (SEQ ID NO: 10), VQDMIQTQV (SEQ ID NO: 11), ILDDWLRHL (SEQ ID NO: 12) and SLPSQHWSL (SEQ ID NO: 13).

31. An expression vector inducing expression of one or more peptide(s) according to claim 30.

32. A cytotoxic T-lymphocyte of a subject treated with one or more peptide(s) according to claim 30, or one or more expression vector(s) inducing expression of said one or more peptide(s).

33. A cytotoxic T-lymphocyte generated in vitro by stimulation of T cells with one or more peptide(s) according to claim 30, or one or more expression vector(s) inducing expression of said one or more peptide(s).

34. An engineered T cell expressing a T-cell receptor recognizing a peptide according to claim 30.

35. A method for treating or preventing at least one cancer in a subject in need thereof, said method comprising the administration of
one or more peptide(s) according to claim 30,
one or more expression vector(s) inducing expression of said one or more peptide(s),
one or more cytotoxic T-lymphocyte(s) of a subject treated with said one or more peptide(s), or with said one or more expression vector(s),
one or more cytotoxic T-lymphocyte(s) generated in vitro by stimulation of T cells with said one or more peptide(s), or with said one or more expression vector(s), or
one or more engineered T cell(s) recognizing one or more peptide(s).
